(19) United States
(12) Patent Application Publication
(10) Pub. No.: US 2004/0057908 A1
(43) Pub. Date: Mar. 25, 2004

(54) Oral compositions and use thereof

(76) Inventors: William H. Bowen, Victor, NY (US); Hyun Koo, Rochester, NY (US); Yong Kun Park, Campinas (BR); Jaime Aparecido Cury, Piracicaba (BR); Pedro Luiz Rosalen, Piracicaba (BR)

Correspondence Address: Edwin V Merkel, Nixon Peabody, Clinton Square, P O Box 31051, Rochester, NY, 14603, US

(21) Appl. No.: 10/450,231

(22) PCT Filed: Dec. 13, 2001
(86) PCT No.: PCT/US01/49032

(51) Int. Cl. A61K 7/16

(52) U.S. Cl. 424/49

(57) ABSTRACT

The present invention relates to an oral composition which includes an organoleptically suitable carrier and an amount of a terpenoid and a flavonoid, dispersed in the carrier, which is effective to prevent or treat dental caries, dental plaque formation, gingivitis, candidiasis, dental stomatitis, aphthous ulceration, or fungal infection. The invention also relates to various uses of oral compositions, containing a terpenoid, a flavonoid, or both, such uses include: inhibiting the activity of surface-bound glusosyltransferase; treating or inhibiting dental caries, gingivitis, candidiasis, denture stomatitis; inhibiting the accumulation of microorganisms on an oral surface; and/or treating or inhibiting aphthous ulcerations on an oral surface.

This application claims the priority benefit of U.S. Provisional Patent Application Serial No. 60/255,304 filed Dec. 13, 2001, which is hereby incorporated by reference in its entirety.

This invention was made, at least in part, with funding received from the National Institutes of Health, NIDCR Grant No. R37 DE07907. The U.S. Government may have certain rights in this invention.

### FIELD OF THE INVENTION

The present invention relates to oral compositions and their use for inhibiting the activity of surface-bound glucosyltransferase; treating or inhibiting dental caries, gingivitis, candidiasis, and/or denture stomatitis; inhibiting the accumulation of microorganisms on an oral surface; and treating or inhibiting aphthous ulcerations on an oral surface.

### BACKGROUND OF TH INVENTION

Colonization of tooth surfaces by mutans streptococci is associated with the etiology and pathogenesis of dental caries in animals and humans (Fitzgerald and Keyes, 1960; Loesche, 1986). Glucosyltransferase enzymes (“GTFs”) produced by Streptococcus mutans have been recognized as virulence factors in the pathogenesis of dental caries (De Stoppelaar et al., 1971; Tanzer et al., 1985; Yamashita et al., 1993). GTFs catalyze the formation of soluble and insoluble α-linked glucans from sucrose and contribute significantly to the polysaccharide composition of dental plaque matrix (Rölla et al., 1983). Dental plaque is essentially a biofilm. Glucans promote the adherence and accumulation of cariogenic streptococci on the tooth surface, and play an essential role in the development of pathogenic dental plaque related to caries activity (Hamada and Slade, 1980; Schilling and Bowen, 1992; Yamashita et al., 1993). Streptococcus mutans produces at least three GTFs: GTF B, which synthesizes a polymer of mostly insoluble α1,3-linked glucan; GTF C, which synthesizes a mixture of insoluble α1,3-linked glucan and soluble α1,6-linked glucan; and GTF D which synthesizes α1,6-linked soluble glucan (Aoki et al., 1986; Hanada and Kuramitsu, 1988; Hanada and Kuramitsu, 1989). An additional enzyme, GTF from S. sanguinis (GTF Ss), may also be involved in the development of dental plaque (Nyvad and Kilian, 1987; Vacca-Smith et al., 2000). S. sanguinis colonizes tooth surface early in plaque formation, and its GTF catalyzes predominantly α1,6-linked soluble glucan (Ceska et al., 1972). Enzymatically active GTFs are present in the soluble fraction of whole human saliva and are also incorporated into salivary pellicle that is formed on tooth surfaces (Rölla et al., 1983; Scheie et al., 1987). Furthermore, the GTFs incorporated into an experimental pellicle demonstrate distinct physical and kinetic properties when compared to the same enzymes in solution; GTF C and D express enhanced enzymatic activity (Schilling and Bowen, 1988; Vacca-Smith et al., 1996; Venkitaraman et al., 1995). A large proportion of the glucans synthesized by these surface-adsorbed GTFs is retained on the pellicle and may provide binding sites for mutans streptococci, contributing to the in situ formation of dental plaque (Schilling and Bowen, 1988; Schilling and Bowen, 1992; Vacca-Smith and Bowen, 1998). Therefore, inhibition of GTFs both in solution and adsorbed to the pellicle of tooth surface is one of the strategies to prevent dental caries and other plaque related diseases.

Propolis, a resinous substance collected by Apis mellifera bees from various plant sources and mixed with secreted beeswax, is a multifunctional material used by bees in the construction, maintenance and protection of their hives (Burdock, 1998; Ghisalberti, 1979). Propolis is a non-toxic natural product with multiple pharmacological effects and a complex chemical composition (Burdock, 1998; Ghisalberti, 1979). Several compounds have been identified in propolis and three distinct chemical groups have been reported to the present: 1) flavonoid aglycones; 2) cinnamic acid derivatives; and 3) terpenoids (Bankova et al., 1995; Banskota et al., 1998; Park et al., 1998; Tazawa et al., 1998). Among them, flavonoids have been considered the main biologically active compounds in propolis (Amoros et al., 1992; Bonhevi et al., 1994; Ghisalberti, 1979). Propolis exhibits a wide range of biological activities, including antimicrobial, anti-inflammatory, anesthetic, and cytostatic properties (Burdock, 1998; Ghisalberti, 1979). It has been demonstrated previously that two chemically distinct types of propolis from Brazil inhibited the activity of GTFs and the growth of mutans streptococci in vitro (Koo et al., 2000a; Koo et al., 2000b; Koo et al., 2000c). Furthermore, topical application twice daily of propolis (Koo et al., 1999) or inclusion in drinking water available ad libitum (Ikeno et al., 1991) reduced the incidence of dental caries in rats. Nevertheless, information on the biological properties of specific compounds, which could be useful in prevention of oral diseases, is sparse. Therefore, it would be desirable to identify individual components of propolis as well as other compounds not in propolis, which have similar activity.

The present invention is directed to overcoming these and other deficiencies in the art.

### SUMMARY OF THE INVENTION

A first aspect of the present invention relates to an oral composition including: an organoleptically suitable carrier and an amount of a terpenoid and a flavonoid, dispersed in the carrier, which is effective to prevent or treat dental caries, dental plaque formation, gingivitis, candidiasis, dental stomatitis, aphthous ulceration, or fungal infections.

A second aspect of the present invention relates to a method of inhibiting the activity of surface-bound glucosyltransferase which includes: contacting a surface-bound glucosyltransferase with an effective amount of a flavonoid or a combination of a flavonoid and a terpenoid, under conditions effective to inhibit the glucan-forming activity of the surface-bound glucosyltransferase.

A third aspect of the present invention relates to a method of treating or inhibiting dental caries, gingivitis, candidiasis, or denture stomatitis, which method includes: providing an oral composition of the present invention and contacting an oral surface with an effective amount of the oral composition under conditions effective to treat or inhibit dental caries, gingivitis, candidiasis, or denture stomatitis.

A fourth aspect of the present invention relates to a method of inhibiting accumulation of microorganisms on an oral surface which includes: providing an oral composition of the present invention and contacting an oral surface with an effective amount of the oral composition under conditions effective to inhibit accumulation of a microorganism which promotes dental caries, gingivitis, candidiasis, denture stomatitis, or formation of dental plaque matrix.

A fifth aspect of the present invention relates to a method of treating or inhibiting aphthous ulceration which includes: contacting an oral surface with an effective amount of a terpenoid, a flavonoid, or a combination thereof, under conditions effective to treat an existing aphthous ulceration or inhibit formation of an aphthous ulceration.

It is believed that the oral compositions of the present invention are particularly well suited for use in treating or inhibiting dental caries, gingivitis, candidiasis, and denture stomatitis, because oral compositions including terpenoids and flavonoids can both disrupt the activity of glucosyltransferases which are in solution and/or bound to a solid surface as well as destroy microorganisms which produce the glucosyltransferases. They are similarly well suited to inhibit accumulation of microorganisms which promote dental caries, gingivitis, candidiasis, denture stomatitis, or formation of dental plaque matrix. Additional benefits include minimizing the staining of teeth and oral malodors.

### BRIEF DESCRIPTION OF THE DRAWINGS

FIG. 1 is a graph illustrating time-kill curves for mutans streptococci strains by tt-farnesol at four times the MIC (or MBC). Streptococcus mutans GS-5 (▴/Δ), S. mutans UA159 (●/∘), S. sobrinus 6715 (▪/□).

FIGS. 2A-B are graphs which illustrate the effects of apigenin on the activities of streptococcal GTFs in solution (2A) and adsorbed onto saliva-coated hydroxyapatite (sHA) surface (2B). GTF B (♦), GTF C (▪), GTF D (Δ), GTF Ss (∘). The data shown are mean values (±SD). The percentage of inhibition was calculated considering the control (DMSO:EtOH, final concentration of 7.5% and 1.25%, vol./vol.) as maximum GTF activity. At each concentration of apigenin, means labeled with symbols (* or δ) are not significantly different from each other, p<0.05.

FIG. 3 is a graph illustrating the effects of apigenin (1.33 mM) on the activities of GTFs in solution and adsorbed onto saliva-coated hydroxyapatite surface. Open bars represent solution assays and marked bars represent surface assays. The percentage of inhibition was calculated considering the control (solute) as 100% GTF activity. tt-Farnesol (1.33 mM), chlorhexidine (1.33 mM), and fluoride (250 ppm) showed either negligible or moderate inhibitory effects.

FIG. 4 is a graph illustrating the effects of 1.33 mM tt-Farnesol and 0.12% CHX (1.33 mM) on the viability of Streptococcus mutans UA159 biofilms. A similar profile was obtained for S. sobrinus 6715. Apigenin (1.33 mM) and fluoride (250 ppm) showed negligible antibacterial activity as measured by the killing of bacteria.

FIG. 5 is a graph illustrating the effects of treatments on smooth-surface caries and severity scores. Values capped by symbols are statistically significantly different from control (p<0.05). ANOVA, comparison for all pairs using Tukey-Kramer HSD.

FIG. 6 is a graph illustrating the effects of treatments on sulcal caries and severity scores. Values capped by symbols are statistically significantly different from control (p<0.05). ANOVA, comparison for all pairs using Tukey-Kramer HSD.

### DETAILED DESCRIPTION OF THE INVENTION

The present invention relates to oral compositions and their use in inhibiting the activity of surface-bound glucosyltransferase; treating or inhibiting dental caries, gingivitis, candidiasis, and/or denture stomatitis; inhibiting the accumulation of microorganisms on an oral surface; and treating or inhibiting aphthous ulcerations on an oral surface.

Oral compositions which can be used in accordance with the present invention include an organoleptically suitable carrier and a terpenoid and/or a flavonoid dispersed in the carrier, preferably both a terpenoid and a flavonoid dispersed in the carrier.

Suitable terpenoids include, without limitation, terpenes, terpinols, diterpenic acids, diterpenes, triterpenes, and derivatives thereof. Exemplary terpenoids include, without limitation, α-farnesol as well as its stereoisomers and derivatives, β-caryophyllene, terpineol, nerolidol, bisabolol, santatol, dehydroabietic acid, abietic acid, β-amyrine, triterpenic alcohol of amyrine, lanosterol, cupressic acid and its derivatives, agathic acid and its derivatives, agathalic acid, betuletol, melliferone, moronic acid, anwuweizonic acid, betulonic acid, syringaldehyde, imbricatoloic acid, communic acid, methyl isocupressate, tremetone, viscidone and its derivatives, α-cadinene, ledol, guajol, α-copaene, β-selinene, α-elemene, calamenene, α-muurolene, γ-muurolene, β-eudesmol, humulene, bulnesol, and combinations thereof. Many such terpenoids are commercially available or readily synthesized according to known procedures.

The terpenoid is present in an amount which is effective to prevent or to treat dental caries, dental plaque formation, gingivitis, candidiasis, dental stomatitis, aphthous ulcerations, and/or fungal infections. Typically, though not exclusively, the effective amount of terpenoid present in the oral composition is less than about 5 percent by weight/volume. Preferably, the terpenoid is present in an amount which is between about 0.01 to about 2 percent by weight/volume, more preferably about 0.01 to about 1.5 percent by weight/volume.

Suitable flavonoids include flavones, flavonols, dihydroflavonols, flavonones, and derivatives thereof. Exemplary flavonoids include, without limitation, apigenin and its derivatives, acacetin, baicalein, chrysin, luteolin, tectochrysin, kaempferol, kaempferide, galangin, isorhamnetin, rhamnetin, myricetin, fisetin, rutin, pinobanksin, pinobanksin-3-acetate, pinobanksin-7-methyl eter, pinocembrin, sakuranetin, isosakuranetin, quercetin, hesperitin, naringin, pinostrobin and its derivatives, trihydroxymethoxyflavanone, tetraxydroxyflavanone, tetrahydroxyflavone, ermanin, 3,5,7-trihydroxy-4′-methoxyflavanol, 5,6,7-trihydroxy-3,4′-dimethoxyflavone, 3,7-dihydroxy-5-methoxyflavanone, 2,5-dihydroxy-7-methoxyflavanone, 3-methylquercetin, 8-methylkaempferol, and combinations thereof. Many such flavonoids are commercially available or readily synthesized according to known procedures.

The flavonoid is present in an amount which is effective to inhibit soluble or surface-bound glucosyltransferase activity or to prevent or to treat dental caries, dental plaque formation, gingivitis, candidiasis, dental stomatitis, aphthous ulcerations, and/or fungal infections. Typically, though not exclusively, the effective amount of flavonoid present in the oral composition is less than about 5 percent by weight/volume. Preferably, the flavonoid is present in an amount which is between about 0.01 to about 1 percent by weight/volume, more preferably about 0.01 to about 0.5 percent by weight/volume.

The oral compositions can take the form of a toothpaste or gel, a powder, a solution (e.g., mouthwash or dental rinse), a suspension, an emulsion, a lozenge, a mucoadhesive vehicle, a tablet or a gum. The composition can also be presented in a delivery vehicle such as a dental floss impregnated with a composition of the present invention.

The particular choice of carrier will depend, at least in part, upon the desired form which the oral composition will take. The carrier is preferably both organoleptically suitable and pharmaceutically acceptable for oral administration. Typically, the carrier will include as a major component one or more of the following: water, glycerin, alcohols such as ethanol, sorbitol, propylene glycol, etc., DMSO, curable polymers, and powders such as starch.

When water is employed, deionized water is preferred. Typically, water can comprise from about 10 to about 85 weight percent of the oral composition, depending upon the formulation.

Polymeric delivery vehicles can include copolymers of polyvinylmethylether with maleic anhydride and other similar delivery enhancing polymers.

According to a preferred embodiment of the oral composition, the oral composition includes effective amounts of both a terpenoid and a flavonoid dispersed in an organoleptically suitable non-polymeric carrier (i.e., substantially free from other propolis components). The terpenoid, flavonoid, and non-polymeric carrier can be of the type described above, present in the amount described above. The molar ratio of terpenoid:flavonoid can be between about 0.1 to about 10:1, preferably about 0.5 to about 5:1.

In addition, the oral composition can include a number of additives, including without limitation, an abrasive agent, a gelling agent, a humectant, a cariostatic agent, a flavoring agent or sweetener, a desensitizing agent, an anti-calculus agent, a whitening agent, a surfactant, a binding agent, a preservative, an opacifying agent, a coloring agent, a buffering agent, or combinations thereof.

Abrasive agents are typically employed in dentifrice compositions. Suitable abrasive agents include, without limitation, aluminum oxide, aluminum hydroxide, calcium hydrogen phosphate dihydrate or anhydride, silica gel, zirconosilicate, silicic anhydride, aluminosilicate, calcium carbonate, calcium pyrophosphate, aluminum silicate, insoluble sodium metaphosphate, magnesium tertiary phosphate, magnesium carbonate, calcium sulfate, synthetic resins, and combinations thereof. Abrasives can generally be employed in effective amounts of between about 20 to about 90 weight percent, more typically about 20 to about 60 weight percent for dentifrices.

Gelling agents (i.e., thickeners) can be used in various compositions to assist in processing. Suitable gelling agents include, without limitation, carrageenan, sodium carboxymethyl cellulose, alkali metal alginates such as sodium alginate, gums, polyvinyl alcohol, and vee gum or the like. Typically, the gelling agents are employed in amount of about 0.3 to about 5 weight percent.

Humectants can also be employed in the oral compositions, particularly toothpastes and gels and oral rinses. Suitable humectants include sorbitol, glycerin, propylene glycol, 1,3-butylene glycol, polyethylene glycol, xylitol, maltitol, lactitol, or the like. The humectant can also be used as the bulk carrier in many instances, in which case it can be present in an amount of about 5 to about 90 weight percent, more typically about 10 to about 60 weight percent.

Cariostatic agents (i.e., non-flavonoid cariostatic agents) can be provided in each form of the oral composition. Suitable cariostatic agents include, without limitation, sodium fluoride, stannous fluoride, aminefluorides, sodium monofluorophosphate, sodium trimeta-phosphate, triclosan, casein, or combinations thereof. If desired, the cariostatic agent can be present in an amount between about 0.01 to about 3 weight percent, more typically between about 0.02 to about 1 weight percent.

Flavoring agents are desired in most oral compositions to enhance the flavor and palatability of the oral composition and, thus, the likelihood of their use. Suitable flavoring agents can be flavoring oils (e.g., oil of spearmint, peppermint, wintergreen, sassafras, clove, sage, eucalyptus, cinnamon, lemon, and orange, methyl salicylate, etc.) or sweeteners (e.g., sucrose, sucralose, lactose, maltose, xylitol, sodium cyclamate, perillartine, aspartyl phenyl alanine methyl ester, saccharine, etc.). Flavoring agents can be present, either individually or collectively, in an amount of about 0.1 to about 10 weight percent, more typically about 0.1 to about 5 weight percent.

Desensitizing agents can be introduced in some embodiments of the oral composition to treat individuals whose teeth are sensitive to thermal shock, chemicals, etc. Suitable desensitizing agents include, without limitation, potassium citrate, potassium chloride, potassium tartrate, potassium bicarbonate, potassium oxalate, potassium nitrate, and strontium salts. Desensitizing agents can be present, either individually or collectively, in an amount of about 0.1 to about 5 weight percent, more typically about 0.1 to about 3 weight percent.

Anti-calculus agents can be introduced in some embodiments of the oral composition to treat tartar formation. Suitable anti-calculus agents include, without limitation, alkali-metal pyrophosphates, hypophosphite-containing polymers, organic phosphonates, phosphocitrates, and combinations thereof. Anti-calculus agents can be present, either individually or collectively, in an amount of about 0.1 to about 5 weight percent, more typically about 0.1 to about 3 weight percent.

Whitening agents can be employed in some forms of the oral composition. Suitable whitening agent including urea peroxide, calcium peroxide, and hydrogen peroxide. Whitening agents can be employed in amounts of about 0.5 to about 5 weight percent.

Surfactants can also be employed in the various oral compositions. Any of a variety of types of surfactants can be utilized, including anionic, nonionic, cationic and zwitterionic or amphoteric surfactants, or combinations thereof. Exemplary anionic surfactants include, without limitation, sodium lauryl sulfate, sodium lauroyl sarcosinate, a-olefin sulfonate, taurate, lauryl monoglyceride sulfate, lauryl monoglyceride sulfonate, and combinations thereof. Exemplary nonionic surfactants include, without limitation, TWEEN, lauroyl diethanol amide, stearyl monoglyceride, sucrose fatty acid esters, lactose fatty acid esters, lactitol fatty acid esters, maltitol fatty acid esters, polyoxyethylene sorbitan monostearate, and combinations thereof. Exemplary ampholytic surfactants include, without limitation, betain and amino acid type surfactants. Surfactants can be present in amount of about 0.5 to about 15 weight percent, more typically about 0.5 to about 10 weight percent.

Binding agents can be utilized, typically for tablet or lozenge forms. Such binding agents include sodium carboxymethyl-cellulose, xanthan gum, gum arabic, etc. as well as synthetic polymers such as polyacrylates and carboxyvinyl polymers. Binders can be present in amounts of about 0.5 to about 50 weight percent depending on the form of the oral composition.

Preservatives can be utilized to enhance the storage properties of the oral composition. One suitable preservative is benzoate (e.g., sodium benzoate), which also possesses a degree of cariostatic activity.

Opacifying agents can also be added to various oral compositions of the present invention. Titanium dioxide is a white powder which adds opacity to the compositions. Titanium dioxide can be present in an amount of about 0.25 to about 5 weight percent.

Coloring agents may also be added to the oral compositions of the present invention. The coloring agent may be in the form of an aqueous solution, i.e., an approximately 1 percent coloring agent in water solution. Color solutions can be present in an amount of about 0.01 to about 5 weight percent.

The oral composition may also include buffers and salts to buffer the pH anionic strength of the oral composition, thereby promoting its stability. The pH of such oral compositions of the invention is generally in the range of about 4.5 to about 9 or 10, preferably about 6.5 to about 7.5 or 8. The pH can be controlled with acid (e.g. citric acid or benzoic acid) or base (e.g. sodium hydroxide) or buffered (as with sodium citrate, benzoate, carbonate, or bicarbonate, disodium hydrogen phosphate, sodium dihydrogen phosphate, etc.).

Preparation of the oral compositions of the present invention can be carried out according to known techniques and procedures, depending upon the particular type of vehicle employed. Where solubility is of concern, suitable surfactants can be employed to enhance the solubility of the active ingredients in the selected carrier. Discussion of the preparation of oral compositions is presented in Harry's Cosmeticology, Seventh Edition, 1982, edited by J. B. Wilkinson and R. J. Moore, published by Chemical Publishing of New York, pages 609 to 617, which is hereby incorporated by reference in its entirety.

It will be understood that, as is conventional, the oral composition are to be packaged according to conventional procedures. Thus a toothpaste or dental cream or gel dentifrice as well as a dental gel will usually be in a collapsible tube or in a squeeze, pump or pressurized dispenser for metering out the contents, having a label describing it, in substance, as a toothpaste, dental cream or the like. A mouth rinse will generally be in a glass or plastic bottle. Lozenges and gum will be packaged individually or in blister packages as is known in the art.

In a preferred use of an oral composition of the present invention, the composition is preferably applied regularly to oral surfaces, such as every day or every second or third day or preferably from two to three times daily, for at least two weeks up to eight weeks or more up to lifetime.

Because of the properties of the flavonoids and/or terpenoids employed in the various oral compositions of the present invention, the oral compositions find numerous uses for improving oral health or inhibiting the decline of oral health. In particular, these aspects of the present invention involve treating or inhibiting dental caries, gingivitis, candidiasis, denture stomatitis, and/or aphthous ulceration, as well as inhibiting accumulation of microorganisms on an oral surface. Basically, these aspects of the present invention are carried out by contacting an oral surface (e.g., a tooth, gum or other mucosal surface, tongue surface, a surface on partial or complete dentures, etc.) with an effective amount of an oral composition of the present invention under conditions effective to achieve the desired effect (i.e., treat or inhibit the above conditions).

A number of oral conditions can be treated, including dental caries, gingivitis, candidiasis, denture stomatitis, and aphthous ulcerations.

Dental caries is one of the most prevalent and significant forms of oral disease which can lead to loss of teeth in spite of development of therapeutics. Caused by dental plaque formed on the surface of teeth, dental caries results in tooth loss as a result of organic acids, the natural metabolite of plaque bacteria contained in dental plaque, which decalcify dental hard tissue locally and gradually. Although fluoride treatments have provided some improvement in public health, there still exists a need to improve the prevention of dental caries. By inhibiting the accumulation of microorganisms responsible for dental caries, such as mutans streptococci, it is possible to inhibit or treat dental caries using the oral compositions of the present invention.

Bacterial plaque on tooth surfaces is also a major etiological factor in gingivitis and periodontitis. Recently, specific oral microorganisms in plaque have been implicated in chronic periodontal disease. One approach to mitigate developing gingivitis and periodontitis is to prevent or reduce plaque formation. The present invention offers an improved approach for the treatment and inhibition of gingivitis by treating oral surfaces with an oral composition of the present invention.

Candidiasis and denture stomatitis are two conditions which are particularly concerned with Candida invasion. Oral candidiasis is seen frequently in two populations, patients wearing dentures and patients who are immunosuppressed or have AIDS.

In the case of the AIDS patients, the oral Candida invasion is usually severe and is one of the earliest signs of human immune deficiency virus infection, occurring prior to the development of opportunistic infections and full blown AIDS (Klein et al., 1984). Presently, there is no known explanation for this localized oral candidiasis, nor is it known whether the oral candidiasis itself plays a role in the development of systemic infections. Because of the severity of infection on the mucosal surface in AIDS patients, antifungal agents are usually prescribed at high dose levels. Such high dosage of antifungals are generally undesirable due to toxic side effects. In addition, treatment failure is often observed.

In the case of the denture stomatitis patients, one of the major problems in treating with prescription antifungals is that the disease recurs soon after treatment with a commercial antifungal is terminated. Investigators have found that this recurrence is due to an inability to destroy C. albicans which adheres to and grows on the acrylic resin surface of the denture (Budtz-Jorgensen, 1974). Therefore, the denture simply reinfects the maxillary palatal mucosal surface to initiate, maintain, and continually aggravate the patient's oral candidiasis infection.

Thus, the oral compositions of the present invention offer an attractive option to treat or inhibit candidiasis and denture stomatitis.

Aphthous ulcerations, commonly referred to as canker sores, are painful oral lesions occurring on the mucous membranes of the tongue, lips, cheek, soft and hard palates, gingiva, floor of the mouth, or pharynx. Several factors have been suggested as possible causes of the disease known as aphthous stomatitis, which is manifested by the formation of aphthous ulcers. However, the disease is still not completely understood, and the factors which cause aphthous stomatitis are still being investigated. Regardless of their cause, the ulcers typically last from 5 to 21 days. Often the ulcers form in groups and merge into a singular extensively ulcerated area. The lesions may become extremely painful, especially during periods of fatigue or during eating, which may become difficult if not impossible. Because of the ability of oral compositions of the present invention to inhibit accumulation of microorganisms on an oral surface, the degree of infection at the site of anaphthous ulcer can be reduced, thereby treating the aphthous ulcer or inhibiting its formation in the first place.

It is believed that the oral compositions of the present invention are particularly well suited for use in treating or inhibiting dental caries, gingivitis, candidiasis, denture stomatitis, and/or aphthous ulceration, because terpenoids and flavonoids, particularly when used in combination, can disrupt the activity of glucosyltransferases which are in solution or bound in a solid surface as well as destroy microorganisms which produce the glucosyltransferases.

The oral compositions of the present invention are also well suited to inhibit accumulation of microorganisms which promote dental caries, gingivitis, candidiasis, denture stomatitis, or formation of dental plaque matrix. Exemplary microorganisms, the accumulation of which can be inhibited, include without limitation: lactobacilli, actinomyces, leptotrichiae, non-β-hemolytic streptococci, enterococci, miscellaneous gram-positive cocci, neisseriae, diphtheroid bacilli, fusiform bacilli, bacteroides, spirochetes, yeasts (Candida), and combinations thereof.

Another use of the oral compositions of the present invention concerns the inhibition of surface-bound glucosyltransferases. It is believed that the present invention for the first time discloses the novel use of flavonoids to inhibit the activity of surface-bound glucosyltransferase, which exhibit different behavior as compared to soluble glucosyltransferase. This aspect of the present invention can be achieved by contacting a surface-bound glucosyltransferase with an effective amount of a flavonoid or a combination of a flavonoid and a terpenoid, under conditions effective to inhibit the glucan-forming activity of the surface-bound glucosyltransferase. Glucosyltransferases which can be inhibited include, without limitation, S. mutans GTF B, S. mutans GTF C, S. mutans GTF D, S. sobrinus GTF, and S. sanguinis GTF.

### EXAMPLES

The following examples are provided to illustrate embodiments of the present invention but are by no means intended to limit its scope.

Materials & Methods

### Test Compounds

The compounds used in this study are classified into three groups: 1) flavonoids (flavonols, flavones, flavanones and dihydroflavonols); 2) cinnamic acid derivatives; and 3) terpenoids. The flavonols (kaempferide, kaempferol, galangin, isorhamnetin, rhamnetin, myricetin, fisetin, rutin), flavones (apigenin, acacetin, baicalein, chrysin, luteolin, tectochrysin), flavanones (pinocembrin, sakuranetin, isosakuranetin), cinnamic acid derivatives (ferulic acid, p-coumaric acid, caffeic acid) and terpenoids (tt-farnesol, β-caryophyllene, terpineol, syringaldehyde) were all obtained from Extrasynthese Co., Genay-Sedex, France. Protocatechuic acid, vanillin, chlorhexidine (CHX), sodium fluoride, and benzoic acid were obtained from Sigma-Aldrich Co., Mass. The dihydroflavonols, pinobanksin, pinobanksin-7-methyl eter, and pinobanksin-3-acetate, were kindly provided by Prof. E. Wollenweber (Darmdstad, Germany).

During animal studies (see Example 2 infra), a single concentration of 1.33 mM (0.035% apigenin and 0.028% tt-farnesol, w/v) was tested along with a positive control 0.12% CHX (equivalent to 1.33 mM).

All the chemical compounds were dissolved in DMSO:ethanol (1:4, v/v) or ethanol (99.9%, HPLC grade) just prior to carrying out the assays. Appropriate solvent controls were always included. Table 1 summarizes all the test compounds used in this study and their chemical structures.

TABLE 1
Chemical structure of compounds used in the study

| Group | Compound | Substitutions | Reference Structure |
|---|---|---|---|
| Flavones | Apigenin Acacetin Baicalein^{a}Chrysin Luteolin^{a}Tectochrysin | 5,7,4′-OH 4′-Me apigenin 5,6,7-OH 5,7-OH 5,7,3′,4′-OH 7-Me chrysin | 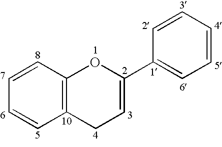 |
| Flavonols | Kaempferol Kaempferide Galangin Isorhamnetin Rhamnetin Myricetin^{a}Fisetin^{a}Rutin | 3,5,7,4′-OH 4′-Me kaempferol 3,5,7-OH 3-Me quercetin 7-Me quercetin 3,5,7,3′,4′,5′-OH 3,7,3′,4′-OH quercetin | |
| # 3-O-rutinoside | 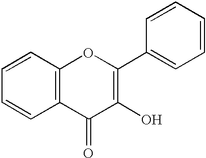 | | |
| Flavanones | Pinocembrin Sakuranetin Isosakuranetin | 5,7-OH 5,4′-OH 7-Me 5,7-OH 4′-Me | 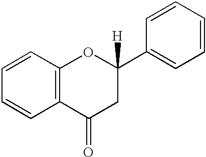 |
| Dihydroflavonols | Pinobanksin Pinobanksin-3-acetate Pinobanksin-7-methyl eter^{a} | 3,5,7-OH 5,7-OH 3-Ac 3,5-OH 7-methyl eter | 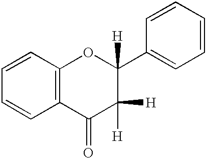 |
| Cinnamic Acid Deriv. | Ferulic acid Caffeic acid p-Coumaric acid | 3-Me 4-OH 3,4-OH 3-H 4-OH | 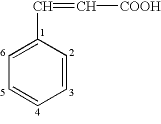 |

| | |
|---|---|
| Others | tt-Farnesol (sesquiterpene), β-Caryophyllene (terpenoid), Terpineol (terpenoid), |
| | Benzoic acid, Syringaldehyde (diterpenic acid), Protocatechuic acid (benzoic acid |
| | deriv.), Vanillin (benzaldehyde deriv.) |

^{a}These compounds were not identified in Apis mellifera propolis.

### Bacterial Strains

The bacterial strains used for the production of GTFs were: 1) Streptococcus milleri KSB8, which harbors the gtfB gene from S. mutans GS-5 (for GTF B production); 2) S. milleri NH5, which contain the gtfD gene S. mutans GS-5 (for GTF D); 3) S. mutans WHB 410 (Wunder and Bowen, 1999), which the genes for GTF B, D and fructosyltransferase were deleted (for GTF C); and 4) S. sanguinis 10904. For susceptibility and time-kill assays, the following bacterial strains were used: 1) S. mutans GS-5; 2) S. mutans UA159; and 3) S. sobrinus 6715. For antibacterial assays, the following bacterial strains were used: 1) S. mutans UA159; and 2) S. sobrinus 6715. The cultures were stored at-80° C. in brain heart infusion (BHI) or tryptic soy broth (TSB) containing 20% glycerol. The S. milleri constructs were a kind gift from Dr. Howard K. Kuramitsu (SUNY, Buffalo, N.Y.) and S. mutans UA159 was obtained from Dr. Robert E. Marquis (Univ. of Rochester, Rochester, N.Y.).

### GTF Enzymes

All the purification procedures were carried out using buffers containing the protease inhibitor phenylmethylsulfonylfluoride-PMSF (1 mM, final concentration) and NaN₃ (0.02%, final concentration) as preservative. Neither of the reagents had any adverse effects on enzyme activity or stability.

The GTFs B, D, and Ss were obtained from culture supernatants and purified to near homogeneity by hydroxyapatite column chromatography as described by Venkitaraman et al. (1995) and Vacca Smith et al. (2000). For GTF C isolation from S. mutans WHB 410 (Wunder and Bowen, 1999), cell pellets were harvested from low molecular weight broth (2.5% tryptone, 1.5% yeast extract, 0.3% glucose, 0.1% fructose and 0.1% sorbitol, which had been ultrafiltered through a 10-kd molecular weight cut-off membrane) cultures of S. mutans WHB 410 (ftf gtfD-gtfB-derivative of S. mutans UA130) grown in dialysis tubing (Schilling and Bowen, 1988). The cells were washed twice in 20 mM potassium phosphate buffer, pH 7.5 containing 1 mM PMSF, and 0.02% sodium azide. The cells were then resuspended in 30 ml of 50 mM potassium phosphate buffer, pH 7.5 containing 0.1% triton X-100, 2M urea, 500 mM NaCl, 0.02% sodium azide and 1 mM PMSF, and incubated at 25° C. for 2 h under gentle agitation. The cell suspension was centrifuged at 10,000 g for 15 min. at 4° C. The supernatant was carefully collected as the source of GTF C and dialyzed against 50 mM potassium buffer, pH 7.5 containing 1 mM PMSF and 0.02% sodium azide. The dialyzed preparation was purified by hydroxyapatite column chromatography as detailed by Venkitaraman et al. (1995).

The purity of the enzyme preparations was analyzed by sodium dodecyl sulfate polyacrylamide gel electrophoresis (SDS-PAGE) in a Hoefer Mighty Small SE245 system (Hoefer Scientific Instruments, San Francisco, Calif., USA) and silver staining (Morrisey, 1981). Pre-stained standards were purchased from BioRad Laboratories. Protein concentration was determined by the method of Lowry et al. (1951), with bovine serum albumin (Sigma Chemical Co., St. Louis, Mo.) used to construct standard curves.

Glucosyltransferase activity was measured by the incorporation of [¹⁴C-glucose] from labeled sucrose (NEN Research Products, Boston, Mass.) into glucans (Germaine et al., 1974; Venkitaraman et al., 1995). The GTF enzyme added to each sample for all assays was equivalent to the amount required to incorporate 1.0-1.5 μmol of glucose over the four hours reaction (1.0-1.5 units).

### Example 1

### Effect of Terpenoids and Flavonoids on GTF Activity in Solution and Adsorbed onto Saliva-coated Hydroxyapatite Surface Assay

For solution assays, purified GTF B, C, D and Ss were mixed with a two-fold dilution series of the test compounds (concentration ranging from 125 to 500 μM) and incubated with [¹⁴C-glucose]-sucrose substrate (0.2 μCi/ml) (200.0 mmol/l sucrose, 40 μmol/l dextran 9000, 0.02% sodium azide in adsorption buffer—50 mM KCl, 1.0 mM KPO₄, 1.0 mM CaCl₂, 0.1 mM MgCl₂−pH=6.5) to reach a final concentration of 100 mmol/l sucrose (200 μl final volume). For the control, the same reaction was carried out, where ethanol:DMSO (final concentration of 7.5% and 1.25%, v/v) or ethanol (final concentration of 5%, v/v) replaced the test agent solutions. The samples were incubated 37° C. with rocking for 4 h. After incubation, ice-cold ethanol (1.0 ml) was added and the samples were stored for 18 h, 4° C. for precipitation of glucans. Radiolabeled glucan was determined by scintillation counting (Germaine et al., 1974; Venkitaraman et al., 1995).

For surface assays, the GTFs were adsorbed to hydroxyapatite beads coated with clarified whole saliva (free of GTF activity), as detailed elsewhere (Koo et al., 2000c; Schilling and Bowen, 1988, Venkitaraman et al., 1995). The saliva-coated hydroxyapatite (sHA) beads were exposed to sufficient enzyme to saturate the surface as determined experimentally. Following the adsorption of the enzyme, the beads were washed 3 times with buffer to remove the loosely bound material and exposed to 300 μl of the two-fold dilution series of test compounds (or control) for 30 min. at the same concentrations described above. The beads were washed and exposed to 300 μl [¹⁴C-glucose]-sucrose substrate (100.0 mmol/l sucrose, final concentration). The radiolabeled glucan formed was collected and quantified by scintillation counting (Germaine et al., 1974; Venkitaraman et al., 1995). All the solution and surface assays were done in quadruplicate in at least 3 different experiments.

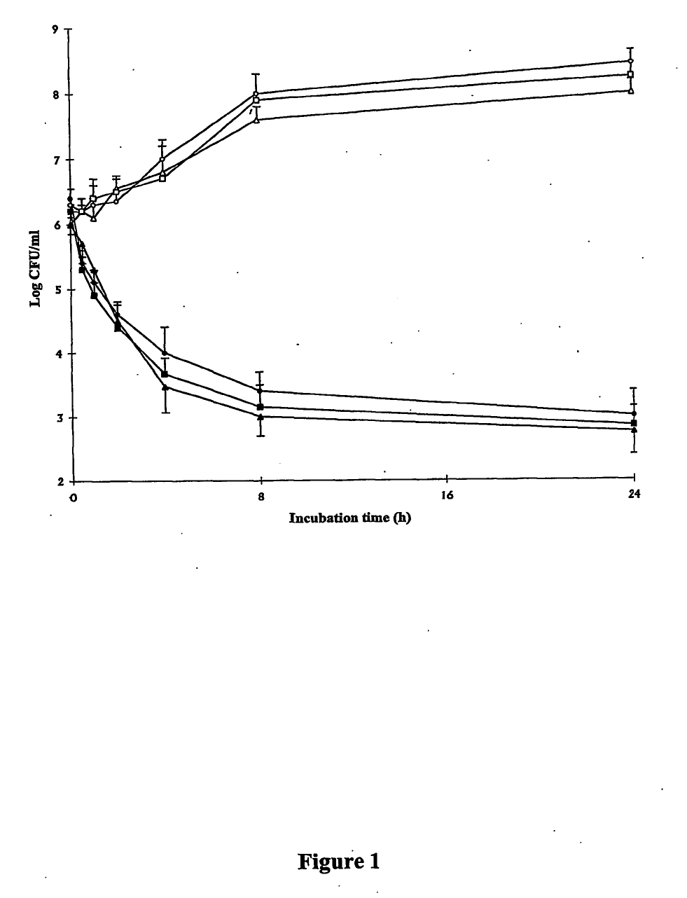

The effects of the most active compounds on the activity of GTFs are shown in the Tables 2 and 3 below. In general, flavonols and flavones reduced the activity of all the enzymes tested in solution (40 to 95% inhibition) and surface (15 to 60% inhibition) at concentration of 500 μM. Among them, apigenin (a 5,7,4′-trihydroxyflavone) displayed the most potent inhibition of GTFs activities. Apigenin inhibited 90.5 to 95% of the activity of all GTFs in solution at a concentration as low as 500 μM (135 μg/ml). The inhibitory effect of apigenin on surface adsorbed enzymes was not as potent as that observed when the same enzymes were in solution. Nevertheless, it was an effective inhibitor (30 to 60% inhibition at a concentration of 500 μM. The inhibitory effects of apigenin on GTFs are illustrated in FIG. 1. Apigenin reduced the activity of all enzymes in solution in direct proportion to the amount added in the reaction test (r² values ranging from 0.92 to 0.99). The IC₅₀ of apigenin (the concentration of test compound required to inhibit the enzymatic activity by 50%) for the GTFs in solution was between 58 (16 μg/ml) to 98 μM (26 μg/ml). The IC₅₀ for surface-adsorbed enzymes were noticeably higher; the IC₅₀ values for GTF B and C were 478 (128 μg/ml) and 458 μM (122 μg/ml); those for GTF D and SS were >1 mM. It is conceivable that an IC₅₀ for this agent would not be achieved for surface-adsorbed GTFs D and Ss. The surface-adsorbed GTF B and C were inhibited significantly more at all concentrations tested than GTF D and Ss (p<0.05). The flavone baicalein, and the flavonols myricetin and rhamnetin also showed to be effective inhibitors of GTFs in solution (70-90% at 500 μM) and adsorbed on saliva-coated hydroxyapatite surface (19-40% at 500 μM).

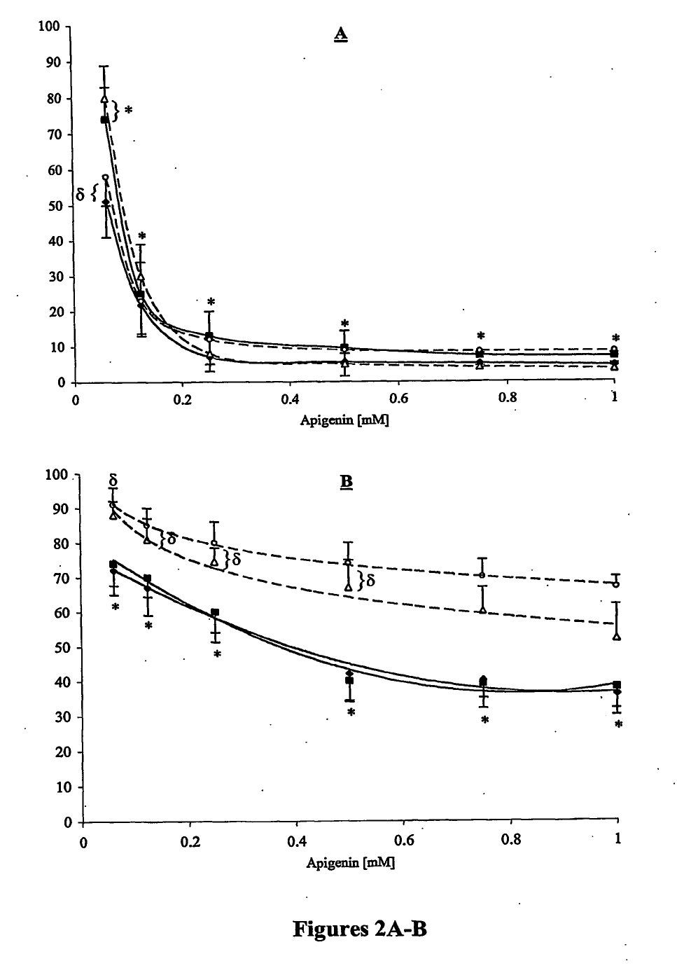

For the compound with highest activity (apigenin), inhibitory curves (concentration-activity; concentrations ranging from 62.5 μM to 1 mM) of all GTFs were plotted and IC₅₀ values (the concentration of test compounds required to inhibit the enzymatic activity by 50%) were calculated from regression lines (Copeland, 2000). For this assay, a one-way layout experimental design was used in 4×2×7 factorial scheme (enzyme×state×dose). An analysis of variance was carried out and qualitative treatments were compared using Tukey test at level of 5% of significance (p<0.05). A non-linear regression was applied in order to evaluate effects of different concentrations. The results are summarized in FIGS. 2A-B.

TABLE 2
Effects of selected flavone and flavonol on the activities of
streptococcal GTFs in solution and adsorbed onto sHA surface^{a}

| | Concen- | GTF B sol. | GTF B surf. | GTF C sol. | GTF C surf. | GTF D sol. | GTF D surf. | GTF Ss sol. | GTF Ss surf. |
|---|---|---|---|---|---|---|---|---|---|
| | tration | % of Inhib. | % of Inhib. | % of Inhib. | % of Inhib. | % of Inhib. | % of Inhib. | % of Inhib. | % of Inhib. |
| Flavone | | | | | | | | | |
| Apigenin | 125 μM | 78.3 (4.8) | 31.8 (8.8) | 77.5 (4.2) | 30.0 (6.4) | 70.8 (4.9) | 17.0 (8.5) | 83.2 (6.1) | 13.8 (2.6) |
| | 250 μM | 92.8 (4.2) | 40.0 (6.3) | 87.5 (3.2) | 42.1 (8.2) | 90.2 (3.6) | 25.5 (3.9) | 91.2 (4.6) | 20.2 (5.4) |
| | 500 μM | 94.3 (3.7) | 55.5 (8.4) | 90.5 (3.8) | 60.5 (5.7) | 95.0 (2.6) | 33.0 (7.9) | 92.7 (3.9) | 30.0 (2.6) |
| Flavonol | | | | | | | | | |
| Kaempferol | 125 μM | 32.4 (5.6) | 9.9 (6.7) | 41.9 (3.2) | 24.2 (4.2) | 39.1 (1.8) | 20.4 (3.3) | 37.6 (5.7) | 9.7 (6.3) |
| | 250 μM | 80.6 (3.5) | 27.8 (2.4) | 82.2 (2.7) | 25.8 (6.7) | 76.7 (5.8) | 23.5 (7.9) | 43.1 (3.3) | 8.8 (3.7) |
| | 500 μM | 90.4 (1.2) | 40.2 (4.5) | 90.2 (2.1) | 35.2 (5.3) | 86.7 (4.2) | 28.5 (5.5) | 88.9 (3.7) | 19.2 (7.2) |

^{a}The data shown are mean values (±SD). The percentage of inhibition was calculated considering the control as maximum GTF activity (100%).

TABLE 3
Effects of selected flavanone and dihydroflavonol on the activities of streptococcal GTFs
in solution and adsorbed onto sHA surface^{a}

| | Concen- | GTF B sol. | GTF B surf. | GTF C sol. | GTF C surf. | GTF D sol. | GTF D surf. | GTF Ss sol. | GTF Ss surf. |
|---|---|---|---|---|---|---|---|---|---|
| | tration | % of Inhib. | % of Inhib. | % of Inhib. | % of Inhib. | % of Inhib. | % of Inhib. | % of Inhib. | % of Inhib. |
| Flavanone | | | | | | | | | |
| Pinocembrin | 125 μM | 3.7 (5.2) | 0.7 (3.3) | 0.0 (0.7) | 0.0 (0.0) | 1.5 (0.1) | 0.0 (0.7) | 25.7 (4.1) | 0.0 (0.2) |
| | 250 μM | 7.2 (1.6) | 8.8 (4.3) | 8.5 (3.2) | 0.4 (0.1) | 4.8 (2.7) | 8.2 (2.7) | 32.6 (6.8) | 0.0 (4.2) |
| | 500 μM | 15.9 (4.7) | 18.9 (5.4) | 17.2 (4.2) | 13.5 (4.9) | 14.9 (4.2) | 15.5 (6.2) | 45.7 (8.3) | 10.5 (2.7) |
| Dihydro- | | | | | | | | | |
| flavonol | | | | | | | | | |
| Pino- | 125 μM | 0.0 (0.8) | 9.4 (4.8) | 0.0 (0.0) | 0.0 (0.1) | 5.5 (4.5) | 0.0 (0.0) | 4.6 (5.7) | 0.0 (0.6) |
| banksin-3- | 250 μM | 7.7 (3.5) | 7.3 (2.1) | 3.5 (4.2) | 0.0 (0.0) | 2.7 (3.3) | 4.5 (2.0) | 22.8 (3.6) | 0.0 (2.7) |
| acetate | 500 μM | 18.9 (2.8) | 10.3 (3.5) | 16.7 (4.3) | 0.0 (3.3) | 3.5 (2.1) | 10.1 (2.8) | 30.5 (7.2) | 9.9 (5.9) |

^{a}The acetate data shown are mean values (±SD). The percentage of inhibition was calculated considering the control as maximum GTF activity (100%).

### Example 2

### Determination of Antibacterial Activity

The minimum inhibitory concentration (MIC) and minimum bactericidal concentration (MBC) were determined for each test compound according to the National Committee for Clinical Laboratory Standards guidelines (Tentative standard M26-T, 1992; NCCLS Publication No. M7-A5, 2000) and Koo et al. (2000b). The broth microdilution and macrodilution methods (in TSB) were used for the antibacterial tests. The starting inoculum was 5×10⁵ CFU/ml and the concentrations of test compounds ranged from 15.6 to 500 μM (two-fold dilutions). The MICs and MBCs were determined in quadruplicate in at least 3 different experiments.

The flavanones, dihydroflavonols, and some terpenoids (tt-farnesol and β-caryophyllene) tested in this study showed moderate inhibitory effects (8-45% for GTFs in solution and 7-24% for GTFs on surface at a concentration of 500 μM); the cinnamic acid derivatives showed negligible effects on GTF enzymes. In some cases the activity of GTFs was enhanced, e.g. by cinnamic acid derivatives and some terpenoids (e.g. protocatechuic acid, terpineol).

The MIC and MBC values of the test compounds for S. mutans (GS-5 and UA159) and S. sobrinus 6715 are shown in Table 4 below. Some of the flavanones and dihydroflavonols, as well as α-farnesol (terpenoid) displayed antibacterial activity. All flavanones inhibited bacterial growth, among them pinocembrin was the most effective with MIC values of 250 μM (64 μg/ml) for all strains tested. Pinocembrin showed bactericidal effect against S. sobrinus 6715 at 500 μM (128 μg/ml). The dihydroflavonol pinobanksin-3-acetate also inhibited the growth of S. sobrinus 6715 and S. mutans strains (MIC values of 500 μM or 157 μg/ml). Among all test compounds, tt-farnesol was the most effective antibacterial agent. The MIC values were 125 μM (28 μg/ml) for S. mutans strains and 62.5 μM (14 μg/ml) for S. sobrinus 6715. The MBC values were 500 μM (112 μg/ml) for S. mutans strains and 250 μM (56 μg/ml) for S. sobrinus 6715. Chlorhexidine (positive control) yielded MIC values between 1.1-2.2 μM (1-2 μg/ml) and MBC values of 8.9. μM (8 μg/ml). Flavonols, flavones and cinnamic acid derivatives did not show any antibacterial activity at concentrations used in this assay, with exception to baicalein (MIC values of 500 μM).

TABLE 4
MIC and MBC values for Antibacterial Compounds Against
Mutans Streptococci Strains^{a}

| | Streptococcus | | Streptococcus | | Streptococcus | |
|---|---|---|---|---|---|---|
| | mutans UA 159 | | mutans GS-5 | | sobrinus 6715 | |
| Test compounds | MIC^{a} | MBC^{a} | MIC | MBC | MIC | MBC |
| Terpenoid | | | | | | |
| tt-Farnesol | 125 | 500 | 125 | 500 | 62.5 | 250 |
| Flavone | | | | | | |
| Baicalein | 500 | >500 | 500 | >500 | 500 | >500 |
| Flavanones | | | | | | |
| Pinocembrin | 250 | >500 | 250 | >500 | 250 | 500 |
| Sakuranetin | >500 | >500 | >500 | >500 | 500 | >500 |
| Isosakuranetin | 500 | >500 | 500 | >500 | 250 | |
| Dihydroflavonol | | | | | | |
| Pinobanksin-3-acetate | 500 | >500 | 500 | >500 | 250 | >500 |

^{a}Values in μM.
Chlorhexidine (positive control) showed MIC values between 1.1-2.2 μM (1-2 μg/ml) and MBC value of 8.9 μM (8 μg/ml).
Kaempferide, kaempferol, galangin, isorhamnetin, rhamnetin, fisetin, rutin, apigenin, acacetin, chrysin, luteolin, tectochrysin, ferulic acid, p-coumaric acid, caffeic acid, β-caryophyllene, terpineol, syringaldehyde, protocatechuic acid, vanillin, benzoic acid, pinobanksin, pinobanksin-7-methyl eter had MICs greater than 500 μM.

For the compound with highest antibacterial activity, the time kill studies were performed by broth macrodilution method (National Committee for Clinical Laboratory Standards, Tentative standard M26-T, 1992). The starting inoculum of S. mutans GS-5 and UA159, and S. sobrinus 6715 was 1-5×10⁶ CFU/ml. The final concentration of the antibacterial agent was 4 times MIC (or MBC values). Tubes containing the microorganisms and the test compound in TSB were incubated in 5% CO₂ at 37° C.; samples were removed for viable counts at 0, 30 min., 1, 2, 4, 8, and 24 h. Serial dilutions (10⁻¹ to 10⁻⁴) were prepared in sterile 0.9% sodium chloride solution. The diluted sample (50 μl) was plated on to tryptic soy agar by means of a spiral plater (Autoplate model 3000, Spiral Biotech, Inc., Bethesda, Md.). The plates were incubated in 5% CO₂ at 37° C. for 48 h, when the number of colonies was determined. Killing curves were constructed by plotting log₁₀ CFU/ml versus time over 24 h. All the assays were done in quadruplicate on at least 3 occasions. Bactericidal effect was defined as a ≧3 log₁₀ decreases in the CFU/ml from the original inoculum. The potential for drug carry-over to produce falsely low viability counts was minimized by dilution of inocula and plating of small volumes of diluted samples (50 μl). In addition, no evidence of drug carry-over was detected at the lowest dilution used for plating (10⁻¹).

The results of the time-kill kinetic studies are summarized in FIG. 1. tt-Farnesol at four times the MIC (or MBC) rapidly reduced the viable counts of mutans streptococci within 30 min. to 1 h of incubation (reduction of 1 log of CFU/ml). tt-Farnesol exerted bactericidal effects (≧3 log decrease in CFU/ml) on S. sobrinus 6715 and on S. mutans strains between 4-8 h incubation. Chlorhexidine at 8.9 μM (MBC) displayed bactericidal effects on mutans streptococci strains tested after 8 h incubation.

Discussion of Examples 1 and 2

Because dental caries results from events that occur at the tooth pellicle-plaque interface, with enzymatically active GTFs present in the pellicle, it is clearly desirable to determine the effects of potential inhibitors on surface-adsorbed GTF.

It has been demonstrated previously that propolis reduced dental caries in desalivated rats (Koo et al., 1999). The present invention, therefore, was directed to the identification of specific compounds in propolis that can, alone and/or in combination, inhibit growth of cariogenic bacteria and the activity of the GTFs, which are associated with the pathogenesis of dental caries and other oral conditions. This was the first step toward identifying novel inhibitors of GTF enzymes and mutans streptococci growth. The results obtained in the present study identified some of the compounds that may have been responsible for the previously reported effects of propolis on GTFs and bacterial growth (Koo et al., 2000a; Koo et al., 2000b; Koo et al., 2000c; Park et al., 1998). In general, flavonoids were the most active compounds, displaying distinct biological properties; flavones and flavonols were effective GTF inhibitors whereas flavanones and the dihydroflavonol pinobanksin-3-acetate showed antibacterial activity.

Apigenin, a 4′,5,7-trihydroxyflavone, was the most effective inhibitor of GTFs, especially GTF B and C. Most of the known GTF inhibitors tested so far, including currently commercially available mouthrinses, failed to inhibit effectively the activities of surface-adsorbed GTFs (Vacca-Smith and Bowen, 1997; Wunder and Bowen, 1999). In contrast, apigenin greatly inhibited GTF, especially GTF B and C, irrespective of whether the enzyme was exposed before or after adsorption to a surface at concentration as low as 500 μM. This level of inhibition has not been observed previously (Vacca-Smith and Bowen, 1997; Wunder and Bowen, 1999). The effective inhibition of GTF B and C by apigenin may affect the pathogenic potential of dental plaque related to caries, consistent with a reduction in smooth-surface caries observed with mutants of mutans streptococci defective in the production of either or both GTFs (Yamashita et al., 1993).

Apigenin is a non-mutagenic flavonoid displaying a variety of anti-tumor and anti-inflammatory effects (Liang et al., 1999; McVean et al., 2000). However, the above data is believed to be the first demonstration of apigenin as a potent inhibitor of GTFs activity. The exact mechanism by which apigenin and related flavonoids act to inhibit the GTFs activity is currently unknown, although the data reported in this study present some insights for understanding the mode of their inhibitory action. Without being bound by theory, it is believed that the inhibition of GTFs depends on the molecular structure of flavonoids and the physical state of the enzyme. Flavones and flavonols, which have an unsaturated double bond between positions C2 and C3 (see Table 1), showed remarkable inhibition of GTFs activity; in contrast, flavanones and dihydroflavonols, which lack double bond in C2-C3, exhibited only modest inhibitory activities. Results from previous studies have shown that flavones and flavonols are the main flavonoids related to inhibition of several mammalian enzymes, which suggest that C2-C3 double bond may be required for maximal inhibitory effects (Eaton et al., 1996; Wheeler and Berry, 1986). The presence of C2-C3 double bond may provide a site for nucleophilic addition by side chains of amino acids in GTFs. Several amino acid residues have been identified as essential for the expression of catalytic activity of GTFs, especially aspartic acid (Kato et al., 1992; Mooser et al., 1991). It is likely that the side chain of aspartic acid (CH₂COOH) may act as a nucleophile and react with flavones and flavonols, causing the GTF inhibition. The resistance displayed by surface-adsorbed GTF enzymes may be related to conformational changes the GTF undergoes during the adsorption process, consistent with differences in physical/kinetic properties and products synthesized between adsorbed and soluble forms of the enzyme (Kopec et al., 1997; Schilling and Bowen, 1988; Venkitaraman et al., 1995).

Several compounds from propolis showed inhibition of mutans streptococci growth. However, none of them was as potent as chlorhexidine, which is a clinically proven antimicrobial. Among the compounds tested, tt-farnesol was the most effective antibacterial agent and displayed a rapid decrease in viable counts of mutans streptococci. This observation is in agreement with previous reports on antimicrobial activity of farnesol and related compounds in variety of other taxa (Bard et al., 1988; Dionigi et al., 1991). Terpenes such as farnesol have been reported to disrupt membrane function, ultimately reducing cell viability (Bard et al., 1988). It is noteworthy that streptococci treated with high concentrations of tt-farnesol (>10 mM) presented visible membrane disruption in the phase-contrast microscope; whether the streptococci membrane was affected at the molecular level by lower concentrations of tt-farnesol (e.g. 0.5 mM) needs to be further elucidated. Recently, farnesol has been shown to be an antifungal agent, displaying quorum-sensing molecule (QSM) activity (Hornby et al., 2001).

The above data supports the hypothesis that the biological activity of propolis, such as caries prevention (Koo et al., 1999), is related to the effects of several compounds, as suggested by Amoros et al. (1992) and Bonhevi et al. (1994), rather than a single compound. Apigenin and tt-farnesol are active compounds in propolis. The concentrations used in this study may be readily achievable in the mouth through topical application. Although details of the toxicology of these compounds were not studied here, there is no evidence in the literature that apigenin or α-farnesol has any potential cellular toxicity or hemolytic effects.

### Example 3

### Antibacterial Assays in Biofilms

To complement the data presented in Examples 1 and 2, biofilms of S. mutans UA159 and S. sobrinus 6715 were used for time-kill studies. Biofilms were formed on standard glass microscope slides in batch cultures for 5 days (Curran et al., 1998). Cells of mutans streptococci were grown in tryptone-yeast extract broth with addition of 1% (w/v) sucrose at 37° C. and 5% CO₂. Typically, 5-day-old biofilms yield approximately 10⁹ colony forming units (CFU) per slide. The killing assays were performed according to Phan et al. (2000). Briefly, 5-day-old biofilms were exposed to test agents (1.33 mM, final concentration) in salt solution (50 mM KCl, 1 mM MGCl₂, pH 7.0) containing DMSO:ethanol (10% and 0.625%, v/v) at 25° C. At specific intervals, the biofilms were removed, suspended in 0.89% NaCl solution, and subjected to sonication by a Branson Sonifier 450 (two times, each three 10-second pulses with 5-second intervals at 50 watts). This sonication procedure provided the maximum recoverable counts as determined experimentally. The homogenized suspension was serially diluted (10¹ to 10⁴) and plated on tryptic soy agar or blood agar by means of a spiral plater (Autoplate model 3000, Spiral Biotech, Inc., Bethesda, Md). The plates were incubated in 5% CO₂ at 37° C. for 48 h, when the number of colonies was determined. Killing curves were constructed by plotting log₁₀ CFU/ml versus time over 4 h. All the assays were carried out in quadruplicate on at least 3 different occasions. Bactericidal effect was defined as a ≧3 log₁₀ decreases in the CFU/ml from initial viable counts, at time zero. The potential for drug carry-over to produce falsely low viability counts was minimized by dilution of inocula and plating of small volumes of diluted samples (50 μl). In addition, drug carry-over was not detected at the lowest dilution used for plating (10¹).

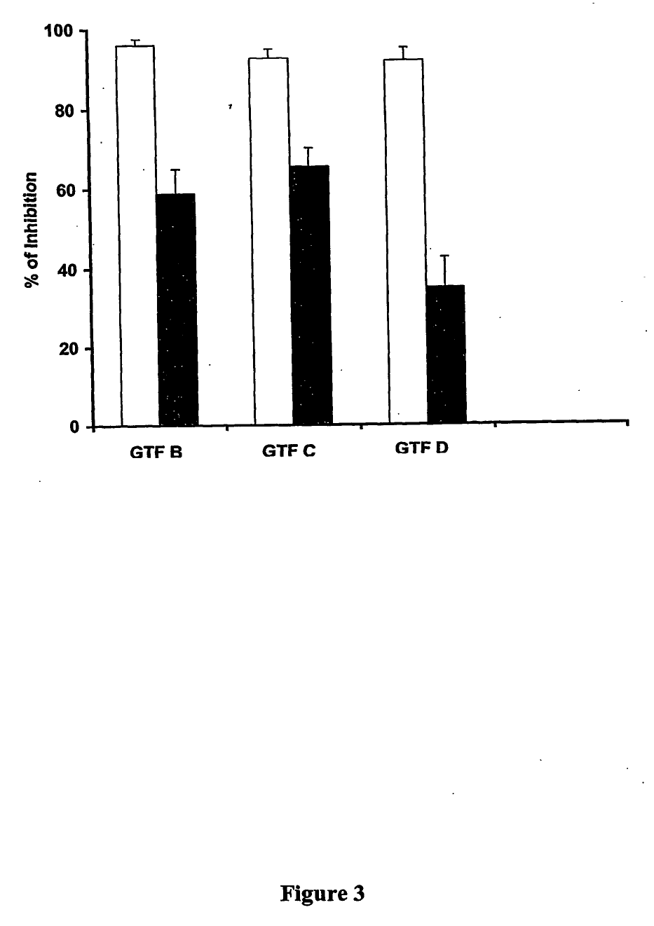

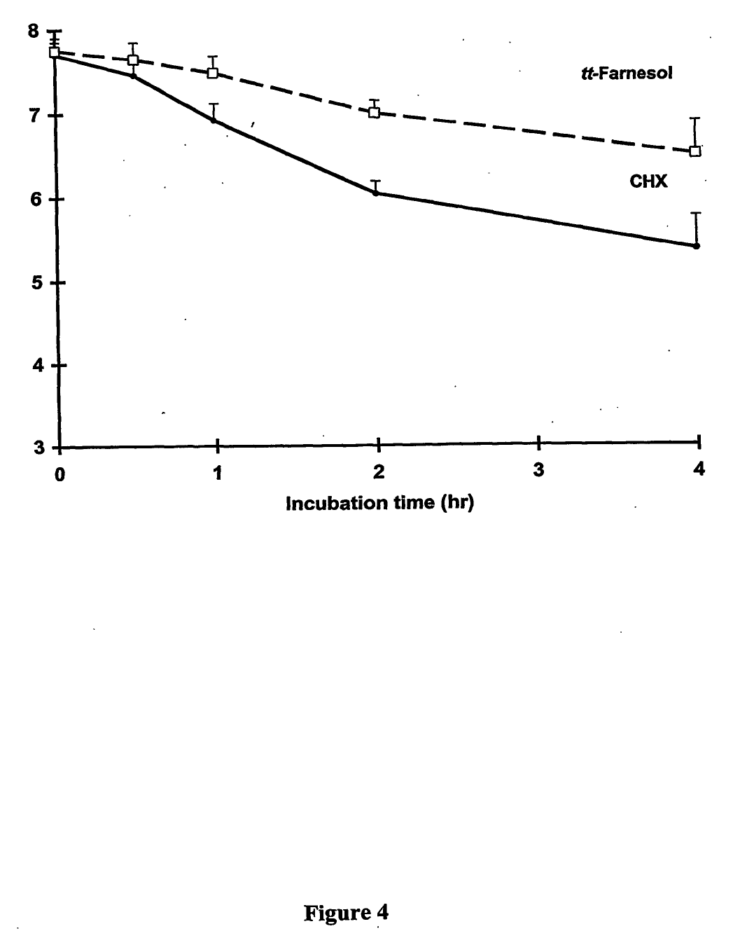

The effects of apigenin on the activity of glucosyltransferases (GTFs) are shown in FIG. 3. Apigenin at 1.33 mM (0.035%) is a potent inhibitor of GTFs whether the enzyme is in solution (90-95% inhibition) or on a surface (60-70%); the effects of tt-farnesol on enzyme activity were negligible (10-20%). Chlorhexidine (CHX) showed only moderate effects (30-45% inhibition in solution and 10-20% on surface). In contrast, both tt-farnesol and CHX showed antibacterial activity against S. mutans and S. sobrinus biofilms as illustrated in FIG. 4. tt-farnesol at 1.33 mM (0.028%) reduced the viable counts of mutans streptococci, showing 1 log₁₀ decrease in CFU/ml after 2-4 h incubation. CHX at 1.33 mM (0.12%) was more effective in reducing the viability of biofilms than was tt-farnesol (2-3 log₁₀ decrease in CFU/mL after same period of incubation). None of the antibacterial agents tested was completely bactericidal against mutans streptococci biofilms. Apigenin was devoid of antibacterial effects against mutans streptococci.

### Example 4

### Inhibition of Dental Caries in Animals

The animal experiment was performed as described previously (Bowen et al., 1988). Seven litters of ten female Sprague Dawley rats aged 14 days were purchased with their dams from Charles River (Kingston, N.Y., USA). Dams were infected using an actively growing overnight culture of Streptococcus sobrinus 6715. At weaning, pups age 21 days were randomly placed into six groups and their teeth treated topically using a camel hair brush twice daily as follows: 1) 0.028% tt-Farnesol (1.33 mM); 2) 0.035% Apigenin (1.33 mM); 3) vehicle control (25% ethanol containing 1.25% DMSO); 4) 250 ppm F; 5) tt-Farnesol+Apigenin (1.33 mM); and 6) 0.12% Chlorhexidine (1.33 mM). Each group of 11 or 12 animals was provided with NIH diet 2000 (Keyes, 1959) (which contains 56% sucrose) and 5% sucrose water ad libitum. The experiment proceeded for five weeks, at which time the animals were killed by CO₂ asphyxiation. The lower left jaw was aseptically dissected, suspended in 5.0 mL of sterile saline solution, and sonicated (three 10-second pulses with 5-second intervals at 30 watts, Branson Sonifier 450). The suspension was plated on mitis salivarius agar plus streptomycin to estimate S. sobrinus population and on blood agar to determine total cultivable flora (TCF). Dental caries was evaluated according to Larson's modification of Keyes' system (Larson, 1981). The determination of caries score was blind by codification of the jaws and was done by 1 calibrated examiner. The data were subjected to ANOVA, the Tukey-Kramer HSD test for all pairs, using software for statistical visualization, JMP version 3.1 (SAS Institute Inc., 1989). Smooth-surface and sulcal caries scores were expressed as proportions of their maximum possible values (124 and 56). The level of significance was 5%.

TABLE 5
Effects of Treatments on Percentage of
Streptococcus sobrinus 6715 in Rats

| Treatments | Infection by S. sobrinus, %* |
|---|---|
| 1.33 mM tt-Farnesol | 44.8 (8.2)^{a} |
| 1.33 mM Apigenin | 40.6 (14.2)^{ab} |
| tt-Farnesol + Apigenin (1.33 mM each) | 31.3 (11.3)^{b} |
| 250 ppm Fluoride | 39.8 (13.2)^{ab} |
| 0.12% Chlorhexidine (1.33 mM) | 34.1 (10.8)^{b} |
| Control | 41.5 (8.4)^{ab} |

* Means (SD): Values followed by the same superscripts are not significantly different from each other (p > 0.05). ANOVA, comparison for all pairs using Tukey-Kramer HSD.

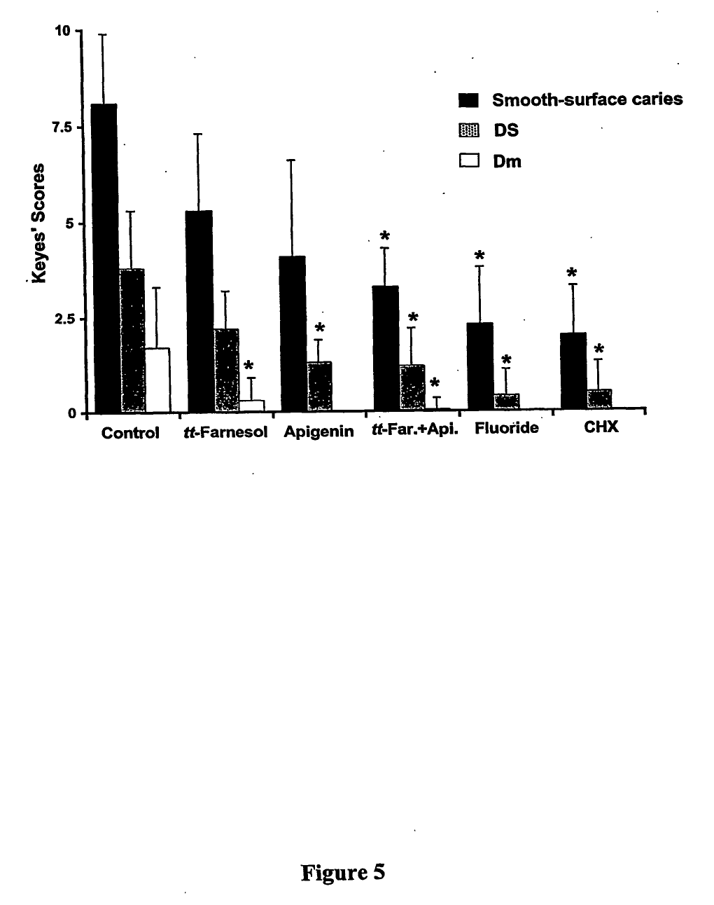

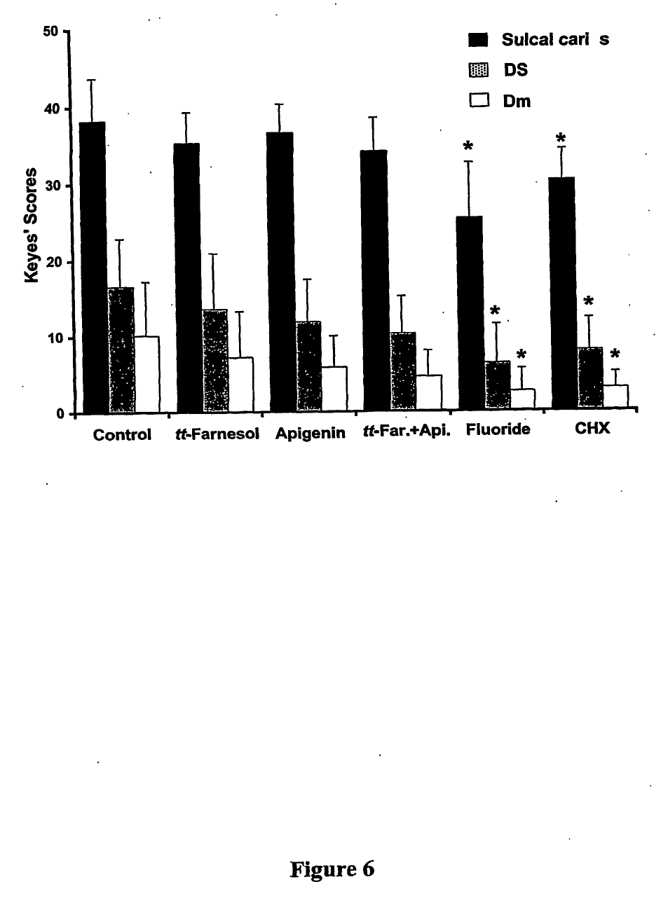

The rats remained in apparent good health during the 5-week experiment. Weight gains were not significantly different among the treatment groups (p>0.05). The effects of the treatments on the incidence and severity of smooth surface caries are shown in FIG. 5. The tt-farnesol+apigenin (60% reduction, Keyes's score: 3.3±3.7), fluoride (72%, 2.3±3.0) and chlorhexidine (75%, 2.0±1.9) groups significantly reduced the incidence of smooth surface caries compared to control group (p<0.05). The smooth-surface caries severity scores were significantly lower in the groups treated with apigenin, apigenin+tt-farnesol, fluoride, chlorhexidine (Ds and Dm levels) and tt-farnesol (Dm) than control (p<0.05). The incidence and severity of sulcal-surface caries were reduced by fluoride and chlorhexidine treatments only (p<0.05) as shown in FIG. 6. The combination of apigenin and tt-farnesol showed significantly better results than individual compounds when compared to control (p<0.05). The percentage of S. sobrinus recovered from the jaws of the rats was calculated from the total cultivable flora and S. sobrinus population. The groups treated with tt-farnesol+apigenin or chlorhexidine showed the lowest levels of infection by S. sobrinus (Table 5), although they did not differ statistically from control group (p>0.05).

Glucosyltransferases synthesize glucan, a capsular-like material, which can protect microorganisms from inimical influences such as antibacterial or antifungal agents, e.g., farnesol. It is believed that the prevention of glucan formation enhances the accessibility of antibacterial or antifungal agents such as farnesol to contact the microorganisms which they are intended to effect, thereby enhancing their efficacy.

The data show clearly that apigenin (0.035%, w/v) and, to a lesser extent, tt-farnesol (0.028%) exhibit a cariostatic effect on smooth caries even at low concentrations. However, neither was as potent as chlorhexidine (0.12%, v/v) and fluoride (250 ppm), which are clinically proven anti-caries agents. Apigenin and tt-farnesol displayed distinct biological properties. The anti-caries mechanism of apigenin may be related to its exceptional inhibitory effects on GTFs activity both in solution and adsorbed onto sHA (FIG. 3). Earlier work has showed that deletion of genes controlling the production of GTFs, especially Gtf B and C, resulted in a dramatic decline in the virulence of S. mutans (Yamashita et al., 1992). Apigenin effectively inhibited the activity of Gtf B and C enzymes and also showed cariostatic properties in the animal based experiment (FIG. 5). This observation is consistent with a reduction in smooth-surface caries observed using mutants of mutans streptococci defective in production of either one or both GTFs (Yamashita et al., 1992). It is likely that apigenin affected the pathogenic potential of dental plaque related to caries by reducing the synthesis of extracellular glucans, because it is devoid of antibacterial effects on mutans streptococci.

Results from a previous study showed that tt-farnesol exhibits bactericidal activity in vitro against planktonic cells of S. sobrinus and S. mutans (Koo et al., 2001), however its effects on mutans streptococci biofilms were less evident (FIG. 4). It has been shown that biofilms are more resistant to antimicrobial agents than cells in suspension (planktonic state), as recently reviewed by Gilbert et al. (1997) and Lewis (2001). The modest effect of it-farnesol (at 1.33 mM) against biofilms may explain its lack of effectiveness to reduce the percentage of S. sobrinus infection in rats. Nevertheless, tt-farnesol was able to reduce the severity of smooth-surface caries at Ds level. Terpenes such as farnesol have been reported to disrupt membrane function, ultimately reducing cell viability (Bard et al., 1988). However, a high concentration of tt-farnesol (<10 mM) would be needed to disrupt the streptococci cells membrane (Koo et al., 2001).

The combination of apigenin and tt-farnesol was more cariostatic than either of the compounds alone. The dynamics of the interaction of apigenin and tt-farnesol in the reduction of the incidence of dental caries is currently unexplored, although the data reported in this study present some insights towards understanding the mode of their inhibitory action. It appears that the inhibition of glucan synthesis by apigenin reduces the biomass of plaque, and also affects the susceptibility of the biofilm to tt-farnesol. This observation could account for the lower level of S. sobrinus infection in rats treated with the combination of apigenin and ft-farnesol compared to those treated with tt-farnesol only (p<0.05) (Table 5). Continued research has demonstrated that apigenin, tt-farnesol, and the combination of apigenin and tt-farnesol inhibits formation of biofilms. Consistent with the results reported above, the combination of apigenin and it-farnesol was more effective than apigenin or tt-farnesol alone.

The positive controls, CHX and fluoride, effectively reduced the incidence of smooth-surface and sulcal surface caries, thereby supporting the validity of the selected model. The anti-plaque effect of CHX has been largely attributed to its antimicrobial activity and oral substantivity; it is a cationic substance that binds to soft and hard tissues of the mouth, as well as to bacterial cell walls (Rolla and Melsen, 1975; Jones, 1997). In the present study, CHX reduced the viability of mutans streptococci biofilms (FIG. 4), although it was not bactericidal. This data confirms the findings in the animal experiment where lower levels of S. sobrinus infection were detected in the group treated with CHX. Fluoride (250 ppm) was devoid of detectable antimicrobial and anti-GTF activity as measured here, although it may reduce acid tolerance and acid production of S. mutans (Marquis, 1990; Belli et al., 1995). Nevertheless, fluoride is the most effective anti-caries agent known to date (Rolla et al., 1991; Clarkson et al., 2000). There is a consensus that the main effect of fluoride is to interfere physicochemically with caries development by reducing demineralization and enhancing remineralization of dental enamel (Dawes and ten Cate, 1990). Thus, substances, which act on virulence factors and/or metabolism of cariogenic bacteria, may increase the anticariogenic effect of fluoride; apigenin could be such an agent.

The above data show clearly that two natural compounds from propolis were cariostatic in the animal model, even at low concentrations. Apigenin was shown to be the first natural cariostatic agent based on its ability to inhibit GTFs; it is a promising anti-caries compound which has a distinct mechanism of action compared to other clinically proven agents.

### LIST OF REFERENCES

Each of the references listed below is hereby incorporated by reference in its entirety into the specification of this application.

Amoros et al., 1992. Synergistic effect of flavones and flavonols against herpes simplex virus type 1 in cell culture. Comparison with the antiviral activity of propolis. J. Nat. Prod. 55:1732-1740.

Aoki et al., 1986. Cloning of a Streptococcus mutans glucosyltransferase gene coding for insoluble glucan synthesis. Infect. Immun. 53:587-594.

Bankova et al., 1995. Chemical composition and antibacterial activity of Brazilian propolis. Z. Naturforsch. 50c:167-172.

Banskota et al., 1998. Chemical constituents of Brazilian propolis and their cytotoxic activities. J. Nat. Prod. 61:896-900.

Bard et al., 1988. Geraniol interferes with membrane functions in strains of Candida and Saccharomyces. Lipids 23:534-538.

Belli et al., 1995. Weak acid effects and fluoride inhibition of glycolysis by Streptococcus mutans GS-5. Can. J. Microbiol. 41(9):785-791.

Bonhevi et al., 1994. The composition, active components and bacteriostatic activity of propolis in dietetics. J. Am. Oil. Chem. Soc. 71:529-532.

Bowen et al., 1988. The effect of desalivation on coronal and root surface caries in rats. J. Dent. Res. 67(1):21-23.

Budtz-Jorgensen, 1974. The significance of Candida albicans in denture stomatitis. Scand. J. Dent. Res. 82:151-190.

Burdock, 1998. Review of the biological properties and toxicity of propolis. Food Chem. Toxicol. 36:341-363.

Ceska et al., 1972. Structural and enzymatic studies on glucans synthesized with glucosyltransferases of some strains of oral streptococci. Acta Chem. Scand. 26:2223-2230.

Clarkson et al., 2000. Role of fluoride in oral health promotion. Int. Dent. J. 50(3):119-28.

Copeland, 2000. In Enzymes: A Practical Introduction to Structure, Mechanism, and Data Analysis 2nd ed. Wiley-VCH, Inc., New York.

Curran et al. 1998. Turning on and turning off the arginine deiminase system in oral streptococci. Can. J. Microbiol. 44:1078-1085.

Dawes and ten Cate, 1990. International symposium on fluorides: Mechanisms of action and recommendation for use. J. Dent. Res. 69(spec. issue).

De Stoppelaar et al., 1971. Decreased cariogenicity of a mutant of Streptococcus mutans. Arch. Oral Biol. 16:971-975.

Dionigi et al., 1991. Effects of farnesol and the off-flavor derivative geosmin on Streptomyces tendae. Appl. Environ. Microbiol. 57:3429-3432.

Eaton et al., 1996. Flavonoids, potent inhibitors of the human p-form phenolsulfotransferase. Potential role in drug metabolism and chemoprevention. Drug Metab. Dispos. 24:232-237.

Fitzgerald and Keyes, 1960. Demonstration of the etiologic role of streptococci in experimental caries in the hamster. J. Am. Dent. Assoc. 61: 9-19.

Germaine et al., 1974. Rapid filter paper assay for the dextransucrase activity from Streptococcus mutans. J. Dent. Res. 53:1355-1360.

Ghisalberti, 1979. Propolis: A review. Bee World 60:59-84.

Gilbert et al., 1997. Biofilm susceptibility to antimicrobials. Adv. Dent. Res. 11(1):160-7.

Hamada and Slade, 1980. Biology, immunology, and cariogenicity of Streptococcus mutans. Microbiol. Rev. 44:331-384.

Hanada and Kuramitsu, 1988. Isolation and characterization of the Streptococcus mutans gtfC gene, coding for synthesis of both soluble and insoluble glucans. Infect. Immun. 56:1999-2005.

Hanada and Kuramitsu, 1989. Isolation and characterization of the Streptococcus mutans gtfD gene, coding for primer-dependent soluble glucan synthesis. Infect. Immun. 57:2079-2085.

Homby et al., 2001. Quorum sensing in the dimorphic fungus Candida albicans is mediated by farnesol. Appl. Environ. Microbiol. 67:2982-2992.

Ikeno et al., 1991. Effects of propolis on dental caries in rats. Caries Res. 25:347-351.

Jones, 1997. Chlorhexidine: Is it still the gold standard? Periodontol. 2000 15:55-62.

Kato et al., 1992. Molecular genetic analysis of the catalytic site of Streptococcus mutans glucosyltransferases. Biochem. Biophys. Res. Commun. 189:1184-1188.

Keyes, 1959. Dental caries in the Syrian hamster. VII Induction of rampant caries activity in albino and golden animals. J. Dent. Res. 38:525-533.

Klein et al., Oral candidiasis in high-risk patients as the initial manifestation of the acquired immunodeficiency syndrome. New Eng. J. Med. 311:354-358 (1984).

Koo et al., 1999. Effect of Apis mellifera propolis from two Brazilian regions on caries development in desalivated rats. Caries Res. 33:393-400.

Koo et al., 2000a. In vitro antimicrobial activity of propolis and Arnica montana against oral pathogens. Arch. Oral Biol. 45:141-148.

Koo et al., 2000b. Effect of a new variety of Apis mellifera propolis on mutans streptococci. Current Microbiol. 41:192-196.

Koo et al., 2000c. Effects of Apis mellifera propolis on the activities of streptococcal glucosyltransferases in solution and adsorbed onto saliva-coated hydroxyapatite. Caries Res. 34:361442.

Kopec et al., 1997. Structural aspects of glucans formed in solution and on the surface of hydroxyapatite. Glycobiol. 7: 929-934.

Larson, 1981. Merits and modification of scoring rat dental caries by Keyes' method, pp. 195-203, In Animal Models in Cariology. Tanzer (ed.), IRL Press, Washington, D.C.

Lewis, 2001. Riddle of biofilm resistance. Antimicrob. Agents Chemother. 45(4):999-1007.

Liang et al., 1999. Suppression of inducible cyclooxygenase and inducible nitric oxide synthase by apigenin and related flavonoids in mouse macrophages. Carcinogenesis 20:1945-1952.

Lowry et al., 1951. Protein measurement with the folin phenol reagent. J. Biol. Chem. 193:265-275.

Loesche, 1986. Role of Streptococcus mutans in human dental decay. Microbiol. Rev. 50: 355-280.

Marquis, 1990. Diminished acid tolerance of plaque bacteria caused by fluoride. J. Dent. Res. 69(Suppl):672-675, 682-683.

McVean et al., 2000. Increase in wild-type p53 stability and transactional activity by the chemopreventive agent apigenin in keratinocytes. Carcinogenesis 21:633-639.

Mooser et al., 1991. Isolation and sequence of an active-site peptide containing a catalytic aspartic acid from two Streptococcus sobrinus α-glucosyltransferases. J. Biol. Chem. 266:8916-8922.

Morrisey, 1981. Silver stain for proteins in polyacrylamide gels: a modified procedure with enhanced uniform sensitivity. Anal. Biochem. 117:307-310.

National Committee for Clinical Laboratory Standards, 1992. Methods for determining bactericidal activity of antimicrobial agents, Tentative standard M26-T. National Committee for Clinical Laboratory Standards, Wayne, Pa.

National Committee for Clinical Laboratory Standards, 2000. Methods for dilution antimicrobial susceptibility tests for bacteria that grow aerobically, 5^{th} ed.

Approved standard. NCCLS publication no. M7-A5. National Committee for Clinical Laboratory Standards, Wayne, Pa.

Nyvad and Kilian, 1987. Microbiology of the early colonization on human enamel and root surfaces in vivo. Scand. J. Dent. Res. 95:369-380.

Park et al., 1998. Antimicrobial activity of propolis on oral microorganisms. Current Microbiol. 36:24-28.

Rölla and Melsen, 1975. On the mechanism of the plaque inhibition by chlorhexidine. J. Dent. Res. 54(Suppl B):B57-62.

Rölla et al., 1983. Free glucosyl- and fructosyltransferase in human saliva and adsorption of these enzymes to teeth in vivo, p. 21-30. In Glucosyltransferases, glucans, sucrose, and dental caries. Chemical Senses, Doyle and Ciardi (eds.), IRL Press, Washington, D.C.

Rölla et al., 1991. Clinical effect and mechanism of cariostatic action of fluoride-containing toothpastes: A review. Int. Dent. J. 41(3):171-174.

Scheie et al., 1987. Glucosyltransferase activity in human in vivo formed pellicle and in whole saliva. Scand. J. Dent. Res. 95:212-215.

Schilling and Bowen, 1988. The activity of glucosyltransferase adsorbed onto saliva-coated hydroxyapatite. J. Dent. Res. 67:2-8.

Schilling and Bowen, 1992. Glucans synthesized in situ in experimental salivary pellicle function as specific binding sites for Streptococcus mutans. Infect. Immun. 60:284-295.

Tanzer et al., 1985. Virulence of mutants defective in glucosyltransferase, dextran-mediated aggregation, or dextranase activity, p. 204-211. In Molecular Basis of Oral Microbial Adhesion, Mergenhagen and Rosan (eds.), American Society for Microbiology, Washington, D.C.

Tazawa et al., 1998. Studies on the constituents of Brazilian propolis. Chem. Pharm. Bull. 46:1477-1479.

Vacca-Smith et al., 1996. Characterization of glucosyltransferase of human saliva adsorbed onto hydroxyapatite surfaces. Caries Res. 30:354-360,

Vacca-Smith and Bowen, 1996. Effects of some antiplaque agents on the activity of glucosyltransferases of Streptococcus mutans adsorbed onto saliva-coated hydroxyapatite and in solution. Biofilm J., Bioline Internat'l (Online Journal).

Vacca-Smith and Bowen, 1998. Binding properties of streptococcal glucosyltransferases for hydroxyapatite, saliva-coated hydroxyapatite, and bacterial surfaces. Arch. Oral Biol. 43:103-110.

Vacca-Smith et al., 2000. Studies concerning the glucosyltransferase of Streptococcus sanguis. Caries Res. 34:295-302.

Venkitaraman et al., 1995. Characterization of glucosyltransferase B, GtfC, and GtfD in solution and on the surface of hydroxyapatite. J. Dent. Res. 74:1695-1701.

Wheeler and Berry, 1986. In vitro inhibition of mouse epidermal cell lipoxygenase by flavonoids: structure-activity relationships. Carcinogenesis 7:33-36.

Wunder and Bowen, 1999. Action of agents on glucosyltransferases from Streptococcus mutans in solution and adsorbed to experimental pellicle. Arch. Oral Biol. 44: 203-214.

Yamashita et al., 1992. Molecular analysis of a Streptococcus mutans strain exhibiting polymorphism in the tandem gtfB and gtfC genes. Infect. Immun. 60(4):1618-1624.

Yamashita et al., 1993. Role of the Streptococcus mutans gtf genes in caries induction in the specific-pathogen-free rat model. Infect. Immun. 61:3811-3817.

Although the invention has been described in detail for the purpose of illustration, it is understood that such detail is solely for that purpose, and variations can be made therein by those skilled in the art without departing from the spirit and scope of the invention which is defined by the following claims.

What is claimed:

1. An oral composition comprising:
an organoleptically suitable carrier; and
an amount of a terpenoid and a flavonoid dispersed in the carrier, which is effective to prevent or treat dental caries, dental plaque formation, gingivitis, candidiasis, dental stomatitis, aphthous ulceration, or fungal infections.

2. The oral composition according to claim 1, wherein the carrier is selected from the group consisting of water, glycerin, alcohol, DMSO, a curable polymer, starch, and a combination thereof.

3. The oral composition according to claim 1, wherein the terpenoid is tt-farnesol or its stereoisomers or derivatives, β-caryophyllene, terpineol, nerolidol, bisabolol, santatol, dehydroabietic acid, abietic acid, β-amyrine, triterpenic alcohol of amyrine, lanosterol, cupressic acid or its derivatives, agathic acid or its derivatives, agathalic acid, betuletol, melliferone, moronic acid, anwuweizonic acid, betulonic acid, syringaldehyde, imbricatoloic acid, communic acid, methyl isocupressate, tremetone, viscidone or its derivatives, δ-cadinene, ledol, guajol, α-copaene, β-selinene, α-elemene, calamenene, α-muurolene, γ-muurolene, β-eudesmol, humulene, bulnesol, or a combination thereof.

4. The oral composition according to claim 1, wherein the flavonoid is apigenin or its derivatives, acacetin, baicalein, chrysin, luteolin, tectochrysin, kaempferol, kaempferide, galangin, isorhamnetin, rhamnetin, myricetin, fisetin, rutin, pinobanksin, pinobanksin-3-acetate, pinobanksin-7-methyl eter, pinocembrin, sakuranetin, isosakuranetin, quercetin, hesperitin, naringin, pinostrobin or its derivatives, trihydroxymethoxy flavanone, tetraxydroxy flavanone, tetrahydroxyflavone, ermanin, 3,5,7-trihydroxy-4′-methoxyflavanol, 5,6,7-trihydroxy-3,4′-dimethoxyflavone, 3,7-dihydroxy-5-methoxyflavanone, 2,5-dihydroxy-7-methoxyflavanone, 3-methylquercetin, 8-methylkaempferol, or a combination thereof.

5. The oral composition according to claim 1, wherein the terpenoid is present in an effective amount of less than about 5 percent by weight/volume.

6. The oral composition according to claim 1, wherein the flavonoid is present in an effective amount of less than about 5 percent by weight/volume.

7. The oral composition according to claim 1, wherein the terpenoid:flavonoid molar ratio is between about 0.1 to about 10:1.

8. The oral composition according to claim 1 further comprising a cariostatic agent.

9. The oral composition according to claim 1 further comprising an abrasive agent, a gelling agent, a humectant, a cariostatic agent, a flavoring agent or sweetener, a desensitizing agent, an anti-calculus agent, a whitening agent, a surfactant, a binding agent, a preservative, an opacifying agent, a coloring agent, a buffering agent, or combinations thereof.

10. The oral composition according to claim 1, wherein the oral composition is a toothpaste or gel, a powder, a solution, a suspension, an emulsion, a lozenge, a mucoadhesive vehicle, a tablet, or a gum.

11. A method of inhibiting the activity of surface-bound glucosyltransferase comprising:
contacting a surface-bound glucosyltransferase with an effective amount of a flavonoid or a combination of a flavonoid and a terpenoid, under conditions effective to inhibit the glucan-forming activity of the surface-bound glucosyltransferase.

12. The method according to claim 11, wherein the surface-bound glucosyltransferase is a S. mutans glucosyltransferase, S. sobrinus glucosyltransferase, or S. sanguinis glucosyltransferase.

13. The method according to claim 11, wherein the flavonoid is apigenin or its derivatives, acacetin, baicalein, chrysin, luteolin, tectochrysin, kaempferol, kaempferide, galangin, isorhamnetin, rhamnetin, myricetin, fisetin, rutin, pinobanksin, pinobanksin-3-acetate, pinobanksin-7-methyl eter, pinocembrin, sakuranetin, isosakuranetin, quercetin, hesperitin, naringin, pinostrobin or its derivatives, trihydroxymethoxy flavanone, tetraxydroxy flavanone, tetrahydroxyflavone, ermanin, 3,5,7-trihydroxy-4′-methoxyflavanol, 5,6,7-trihydroxy-3,4′-dimethoxyflavone, 3,7-dihydroxy-5-methoxyflavanone, 2,5-dihydroxy-7-methoxyflavanone, 3-methylquercetin, 8-methylkaempferol, or a combination thereof.

14. The method according to claim 11 wherein said contacting is carried out on an oral surface.

15. The method according to claim 14, wherein the oral surface is a tooth, a mucosal surface, a tongue surface, a surface on complete or partial dentures, or a combination thereof.

16. The method according to claim 11, wherein said contacting is carried out at least twice daily.

17. The method according to claim 11, wherein the flavonoid or the combination of the flavonoid and the terpenoid are present in an oral composition.

18. The method according to claim 17, wherein the oral composition is in the form of a toothpaste or gel, a powder, a solution, a suspension, an emulsion, a lozenge, a mucoadhesive vehicle, a tablet, or a gum.

19. The method according to claim 11, wherein the effective amount of the flavonoid is less than about 5 percent by weight/volume.

20. A method of treating or inhibiting dental caries, gingivitis, candidiasis, or denture stomatitis, said method comprising:
providing an oral composition according to claim 1 and
contacting an oral surface with an effective amount of the oral composition under conditions effective to treat or inhibit dental caries, gingivitis, candidiasis, or denture stomatitis.

21. The method according to claim 20, wherein the oral surface is a tooth, a mucosal surface, a tongue surface, a surface on complete or partial dentures, or a combination thereof.

22. The method according to claim 20, wherein said contacting is carried out at least twice daily.

23. The method according to claim 20, wherein the terpenoid is tt-farnesol or its stereoisomers or derivatives, β-caryophyllene, terpineol, nerolidol, bisabolol, santatol, dehydroabietic acid, abietic acid, β-amyrine, triterpenic alcohol of amyrine, lanosterol, cupressic acid or its derivatives, agathic acid or its derivatives, agathalic acid, betuletol, melliferone, moronic acid, anwuweizonic acid, betulonic acid, syringaldehyde, imbricatoloic acid, communic acid, methyl isocupressate, tremetone, viscidone or its derivatives, δ-cadinene, ledol, guajol, α-copaene, β-selinene, α-elemene, calamenene, α-muurolene, γ-muurolene, β-eudesmol, humulene, bulnesol, or a combination thereof.

24. The method according to claim 20, wherein the flavonoid is apigenin or its derivatives, acacetin, baicalein, chrysin, luteolin, tectochrysin, kaempferol, kaempferide, galangin, isorhamnetin, rhamnetin, myricetin, fisetin, rutin, pinobanksin, pinobanksin-3-acetate, pinobanksin-7-methyl eter, pinocembrin, sakuranetin, isosakuranetin, quercetin, hesperitin, naringin, pinostrobin or its derivatives, trihydroxymethoxy flavanone, tetraxydroxy flavanone, tetrahydroxyflavone, ermanin, 3,5,7-trihydroxy-4′-methoxyflavanol, 5,6,7-trihydroxy-3,4′-dimethoxyflavone, 3,7-dihydroxy-5-methoxyflavanone, 2,5-dihydroxy-7-methoxyflavanone, 3-methylquercetin, 8-methylkaempferol, or a combination thereof.

25. The method according to claim 20, wherein the terpenoid is present in an effective amount of less than about 5 percent by weight/volume.

26. The method according to claim 20, wherein the flavonoid is present in an effective amount of less than about 5 percent by weight/volume.

27. The method according to claim 20, wherein the terpenoid:flavonoid molar ratio is between about 0.1 to about 10:1.

28. The method according to claim 20, wherein the oral composition is in the form of a toothpaste or gel, a powder, a solution, a suspension, an emulsion, a lozenge, a mucoadhesive vehicle, a tablet, or a gum.

29. The method according to claim 20, wherein the oral composition further comprises a cariostatic agent.

30. A method of inhibiting accumulation of microorganisms on an oral surface comprising:
providing an oral composition according to claim 1 and
contacting an oral surface with an effective amount of the oral composition under conditions effective to inhibit accumulation of a microorganism which promotes dental caries, gingivitis, candidiasis, denture stomatitis, or formation of dental plaque matrix.

31. The method according to claim 30, wherein the oral surface is a tooth, a mucosal surface, a tongue surface, a surface on complete or partial dentures, or a combination thereof.

32. The method according to claim 30, wherein said contacting is carried out at least twice daily.

33. The method according to claim 30, wherein the terpenoid is tt-farnesol or its stereoisomers or derivatives, β-caryophyllene, terpineol, nerolidol, bisabolol, santatol, dehydroabietic acid, abietic acid, α-amyrine, triterpenic alcohol of amyrine, lanosterol, cupressic acid or its derivatives, agathic acid or its derivatives, agathalic acid, betuletol, melliferone, moronic acid, anwuweizonic acid, betulonic acid, syringaldehyde, imbricatoloic acid, communic acid, methyl isocupressate, tremetone, viscidone or its derivatives, δ-cadinene, ledol, guajol, α-copaene, β-selinene, α-elemene, calamenene, α-muurolene, γ-muurolene, β-eudesmol, humulene, bulnesol, or a combination thereof.

34. The method according to claim 30, wherein the flavonoid is apigenin or its derivatives, acacetin, baicalein, chrysin, luteolin, tectochrysin, kaempferol, kaempferide, galangin, isorhamnetin, rhamnetin, myricetin, fisetin, rutin, pinobanksin, pinobanksin-3-acetate, pinobanksin-7-methyl eter, pinocembrin, sakuranetin, isosakuranetin, quercetin, hesperitin, naringin, pinostrobin or its derivatives, trihydroxymethoxy flavanone, tetraxydroxy flavanone, tetrahydroxyflavone, ermanin, 3,5,7-trihydroxy-4′-methoxyflavanol, 5,6,7-trihydroxy-3,4′-dimethoxyflavone, 3,7-dihydroxy-5-methoxyflavanone, 2,5-dihydroxy-7-methoxyflavanone, 3-methylquercetin, 8-methylkaempferol, or a combination thereof.

35. The method according to claim 30, wherein the terpenoid is present in an effective amount of less than about 5 percent by weight/volume.

36. The method according to claim 30, wherein the flavonoid is present in an effective amount of less than about 5 percent by weight/volume.

37. The method according to claim 30, wherein the terpenoid:flavonoid molar ratio is between about 0.1 to about 10:1.

38. The method according to claim 30, wherein the oral composition is in the form of a toothpaste or gel, a powder, a solution, a suspension, an emulsion, a lozenge, a mucoadhesive vehicle, a tablet, or a gum.

39. The method according to claim 30, wherein the oral composition further comprises a cariostatic agent.

40. The method according to claim 30, wherein the microorganism is selected from the group consisting of lactobacilli, actinomyces, leptotrichiae, non-β-hemolytic streptococci, enterococci, miscellaneous gram-positive cocci, Neisseriae, diphtheroid bacilli, fusiform bacilli, bacteroides, spirochetes, yeasts, and combinations thereof.

41. A method of treating or inhibiting aphthous ulceration comprising:
contacting an oral surface with an effective amount of a terpene, a flavonoid, or a combination thereof, under conditions effective to treat an existing aphthous ulceration or inhibit formation of anaphthous ulceration.

42. The method according to claim 41, wherein the oral surface is a tooth, a mucosal surface, a tongue surface, complete or partial dentures, or a combination thereof.

43. The method according to claim 41, wherein said contacting is carried out at least twice daily.

44. The method according to claim 41, wherein the terpenoid is α-farnesol or its stereoisomers or derivatives, β-caryophyllene, terpineol, nerolidol, bisabolol, santatol, dehydroabietic acid, abietic acid, β-amyrine, triterpenic alcohol of amyrine, lanosterol, cupressic acid or its derivatives, agathic acid or its derivatives, agathalic acid, betuletol, melliferone, moronic acid, anwuweizonic acid, betulonic acid, syringaldehyde, imbricatoloic acid, communic acid, methyl isocupressate, tremetone, viscidone or its derivatives, δ-cadinene, ledol, guajol, α-copaene, β-selinene, α-elemene, calamenene, α-muurolene, γ-muurolene, β-eudesmol, humulene, bulnesol, or a combination thereof.

45. The method according to claim 41, wherein the effective amount of the terpenoid is less than about 5 percent by weight/volume.

46. The method according to claim 41, wherein the flavonoid is apigenin or its derivatives, acacetin, baicalein, chrysin, luteolin, tectochrysin, kaempferol, kaempferide, galangin, isorhamnetin, rhamnetin, myricetin, fisetin, rutin, pinobanksin, pinobanksin-3-acetate, pinobanksin-7-methyl eter, pinocembrin, sakuranetin, isosakuranetin, quercetin, hesperitin, naringin, pinostrobin or its derivatives, trihydroxymethoxy flavanone, tetraxydroxy flavanone, tetrahydroxyflavone, ermanin, 3,5,7-trihydroxy-4′-methoxyflavanol, 5,6,7-trihydroxy-3,4′-dimethoxyflavone, 3,7-dihydroxy-5-methoxyflavanone, 2,5-dihydroxy-7-methoxyflavanone, 3-methylquercetin, 8-methylkaempferol, or a combination thereof.

47. The method according to claim 41, wherein the effective amount of the flavonoid is less than about 5 percent by weight/volume.

48. The method according to claim 41, wherein the combination of the terpenoid and the flavonoid is provided, the combination being in the form of an oral composition.

49. The method according to claim 48, wherein the terpenoid:flavonoid molar ratio is between about 0.1 to about 10:1.

50. The method according to claim 48, wherein the oral composition is in the form of a toothpaste or gel, a powder, a solution, a suspension, an emulsion, a lozenge, a mucoadhesive vehicle, a tablet, or a gum.
